SEQUENCE LISTING (35 sequences)

SEQ ID NO: 1 - protein, 214 aa, synthetic construct
  REGION 1..214: note = artificial sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
  REGION 1..450: note = artificial sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 694 aa, synthetic construct
  REGION 1..694: note = artificial sequence
VQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIQMTQSPSSLSASVGDRVTITCRASQDVGSGVAWYQEKPGKAPKLLIGSASGLYSGVPSRFSGSRSGTDFTLTISSLQPEDFATYYCQQSASGGSTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - protein, 701 aa, synthetic construct
  REGION 1..701: note = artificial sequence
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGEVQLVESGGGLVQPGGSLRLSCAASGFSIAGTAIHWVRKAPGKGLEWVASISPGGGSTAYADSVKGRFTISADTSKNTAYLQMNSLRAEDTAVYYCSRSGGSGASAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 214 aa, synthetic construct
  REGION 1..214: note = artificial sequence
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, synthetic construct
  REGION 1..449: note = artificial sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
  REGION 1..214: note = artificial sequence
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, synthetic construct
  REGION 1..450: note = artificial sequence
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 453 aa, synthetic construct
  REGION 1..453: note = artificial sequence
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 212 aa, synthetic construct
  REGION 1..212: note = artificial sequence
DIQMTQSPSSLSASVGDRVTITCRASQDVGSGVAWYQEKPGKAPKLLIGSASGLYSGVPSRFSGSRSGTDFTLTISSLQPEDFATYYCQQSASGGSTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 11 - protein, 444 aa, synthetic construct
  REGION 1..444: note = artificial sequence
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 227 aa, synthetic construct
  REGION 1..227: note = artificial sequence
EVQLVESGGGLVQPGGSLRLSCAASGFSIAGTAIHWVRKAPGKGLEWVASISPGGGSTAYADSVKGRFTISADTSKNTAYLQMNSLRAEDTAVYYCSRSGGSGASAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 13 - protein, 695 aa, synthetic construct
  REGION 1..695: note = artificial sequence
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIQMTQSPSSLSASVGDRVTITCRASQDIKNYLNWYQEKPGKAPKLLIYYSSTLLSGVPSRFSGSGSGTDFTLTISSLQPEDFATYYCQQSITLPPTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 14 - protein, 706 aa, synthetic construct
  REGION 1..706: note = artificial sequence
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGQVQLVESGGGLVKPGGSLRLSCAASGFTFSDYAMSWIRKAPGKGLEWVSSINIGATYIYYADSVKGRFTISRDNAKNSLYLQMNSLRAEDTAVYYCARPGSPYEYDKAYYSMAYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 15 - protein, 706 aa, synthetic construct
  REGION 1..706: note = artificial sequence
QVQLVESGGGLVKPGGSLRLSCAASGFTFSDYAMSWIREAPGKGLEWVSSINIGATYIYYADSVKGRFTISRDNAKNSLYLQMNSLRAEDTAVYYCARPGSPYEYDKAYYSMAYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 16 - protein, 695 aa, synthetic construct
  REGION 1..695: note = artificial sequence
DIQMTQSPSSLSASVGDRVTITCRASQDIKNYLNWYQKKPGKAPKLLIYYSSTLLSGVPSRFSGSGSGTDFTLTISSLQPEDFATYYCQQSITLPPTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGEVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 17 - protein, 701 aa, synthetic construct
  REGION 1..701: note = artificial sequence
EVQLVESGGGLVQPGGSLRLSCAASGFSIAGTAIHWVREAPGKGLEWVASISPGGGSTAYADSVKGRFTISADTSKNTAYLQMNSLRAEDTAVYYCSRSGGSGASAMDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 18 - protein, 695 aa, synthetic construct
  REGION 1..695: note = artificial sequence
DIQMTQSPSSLSASVGDRVTITCRASQDVGSGVAWYQKKPGKAPKLLIGSASGLYSGVPSRFSGSRSGTDFTLTISSLQPEDFATYYCQQSASGGSTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGEVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 19 - protein, 700 aa, synthetic construct
  REGION 1..700: note = artificial sequence
EVQLVQSGAEVKKPGASVKVSCKASGYTFTNYYIHWVREAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGGGSGGSGGDIQMTQSPSSLSASVGDRVTITCHASQNIYVWLNWYQEKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 706 aa, synthetic construct
  REGION 1..706: note = artificial sequence
DIVMTQSPDSLAVSLGERATINCKSSQSLLNSRTRKNYLAWYQKKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGGGSGGSGGQVQLVQSGAEVKKPGASVKVSCKASGYTFTSYYIHWVRKAPGQGLEWIGCIYPGNVNTNYNEKFKDRATLTVDTSISTAYMELSRLRSDDTAVYFCTRSHYGLDWNFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 701 aa, synthetic construct; identical to SEQ ID NO: 17
  REGION 1..701: note = artificial sequence

SEQ ID NO: 22 - protein, 695 aa, synthetic construct; identical to SEQ ID NO: 18
  REGION 1..695: note = artificial sequence

SEQ ID NO: 23 - protein, 706 aa, synthetic construct
  REGION 1..706: note = artificial sequence
QVQLVQSGAEVKKPGASVKVSCKASGYTFTSYYIHWVREAPGQGLEWIGCIYPGNVNTNYNEKFKDRATLTVDTSISTAYMELSRLRSDDTAVYFCTRSHYGLDWNFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGGGSGGSGGDIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 700 aa, synthetic construct
  REGION 1..700: note = artificial sequence
DIQMTQSPSSLSASVGDRVTITCHASQNIYVWLNWYQKKPGKAPKLLIYKASNLHTGVPSRFSGSGSGTDFTLTISSLQPEDFATYYCQQGQTYPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGGGSGGSGGEVQLVQSGAEVKKPGASVKVSCKASGYTFTNYYIHWVRKAPGQGLEWIGWIYPGDGNTKYNEKFKGRATLTADTSTSTAYLELSSLRSEDTAVYYCARDSYSNYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 218 aa, synthetic construct
  REGION 1..218: note = artificial sequence
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 450 aa, synthetic construct
  REGION 1..450: note = artificial sequence
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 694 aa, synthetic construct
  REGION 1..694: note = artificial sequence
QVQLKQSGPGLVQPSQSLSITCTVSGFSVTSYGVHWIRESPGKGLEWLGVIWSGGSTDYNAAFISRLTISKDNSKSQVFFKVNSLQPADTAIYYCARAGDYNYDGFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIQMTQSPSTLSASVGDRVTITCRASQSISSWLAWYQEKPGKAPKLLIYDASSLESGVPSRFSGSGSGTEFTLTISSLQPDDFATYYCQQYSSYYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 28 - protein, 699 aa, synthetic construct
  REGION 1..699: note = artificial sequence
QVQLVQSGAEVKKPGASVKVSCKASGYTFTSYYMHWVREAPGQGLEWMGIINPSGGSTSYAQKFQGRVTMTRDTSTSTVYMELSSLRSEDTAVYYCARDYSWWGDSYTGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIQMTQSPSTLSASVGDRVTITCRASQSISSWLAWYQEKPGKAPKLLIYDASSLESGVPSRFSGSGSGTEFTLTISSLQPDDFATYYCQQYNSDWWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 29 - protein, 698 aa, synthetic construct
  REGION 1..698: note = artificial sequence
QVQLVQSGAEVKKPGASVKVSCKASGYTFTSYYMHWVREAPGQGLEWMGIINPSGGSTSYAQKFQGRVTMTRDTSTSTVYMELSSLRSEDTAVYYCARDYSWWGDSYTGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESGGGGSGGGGSGGGGSGGGGSGGGGSGGSGGDIQMTQSPSTLSASVGDRVTITCRASQSISSWLAWYQEKPGKAPKLLIYDASSLESGVPSRFSGSGSGTEFTLTISSLQPDDFATYYCQQYSSYYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 30 - protein, 214 aa, synthetic construct
  REGION 1..214: note = artificial sequence
DIQMTQSPSSLSASVGDRVTITCRASQSISSYLNWYQQKPGKAPKLLIYTASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 454 aa, synthetic construct
  REGION 1..454: note = artificial sequence
EVQLLESGGGLVQPGGSLRLSCAASGFTFSSYAMSWVRQAPGKGLEWVSVITGSGGSTYYADSVKGRFTISRDNSRNTLYLQMNSLRAEDTAVYYCAKGEGYAGSSYFRASDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 692 aa, synthetic construct
  REGION 1..692: note = artificial sequence
QIVLTQSPAIMSASPGEKVTMTCSGSSSVSFMYWYQKRPGSSPRLLIYDTSNLASGVPVRFSGSGSGTSYSLTISRMEAEDAATYYCQQWSTYPLTFGAGTKLELKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGEVQLLESGGGLVQPGGSLRLSCAASGFTFSSYAMSWVRKAPGKGLEWVSAISGSGGSTYYADSVKGRFTISRDNSKNTLYLQMNSLRAEDTAVYYCARLLYYGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 33 - protein, 705 aa, synthetic construct
  REGION 1..705: note = artificial sequence
DIVMTQSPLSLPVTPGEPASISCRSSQSLLHSNGYNYLDWYLKKPGQSPQLLIYLGSNRASGVPDRFSGSGSGTDFTLKISRVEAEDVGVYYCMQVLQPPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGQVQLVQSGAEVKKPGSSVKVSCKASGGTFSSYAISWVRKAPGQGLEWMGGIIPIFGTANYAQKFQGRVTITADKSTSTAYMELSSLRSEDTAVYYCARDPGPPYNWYWGAYDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 34 - protein, 698 aa, synthetic construct
  REGION 1..698: note = artificial sequence
DIVMTQSPLSLPVTPGEPASISCRSSQSLLHSNGYNYLDWYLKKPGQSPQLLIYLGSNRASGVPDRFSGSGSGTDFTLKISRVEAEDVGVYYCMQVLQPPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSGGGGSGGGGSGGGGSGGGGSGGGGSGGSGGEVQLLESGGGLVQPGGSLRLSCAASGFTFSSYAMSWVRKAPGKGLEWVSAISGSGGSTYYADSVKGRFTISRDNSKNTLYLQMNSLRAEDTAVYYCARLLYYGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGESDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 35 - protein, 5 aa, synthetic construct
GGGGS

(19) United States
(12) Patent Application Publication
(10) Pub. No.: US 2026/0008869 A1
(43) Pub. Date: Jan. 8, 2026

(54) RECOMBINANT BINDING PROTEINS WITH ACTIVATABLE EFFECTOR DOMAIN

(71) Applicant: Hoffmann-La Roche Inc., Little Falls, NJ (US)

(72) Inventors: Ulrich BRINKMANN, Welheim (DE); Can Martin BULDUN, München (DE); Steffen DICKOPF, Penzberg (DE); Vedran VASIC, München (DE)

(73) Assignee: Hoffmann-La Roche Inc., Little Falls, NJ (US)

(21) Appl. No.: 19/207,270

(22) Filed: May 13, 2025

Related U.S. Application Data
(63) Continuation of application No. PCT/EP2023/081671, filed on Nov. 14, 2023.

(30) Foreign Application Priority Data
Nov. 15, 2022 (EP) 22207437.9

(51) Int. Cl. C07K 16/32; C07K 16/28

(52) U.S. Cl. CPC C07K 16/32; C07K 16/2809; C07K 16/2818; C07K 16/2863; C07K 2317/31; C07K 2317/522; C07K 2317/526; C07K 2317/55

(57) ABSTRACT

The present invention relates to a pair of recombinant binding proteins and its uses, e.g. for activation of an effector domain upon binding to a target cell. Specifically, the invention relates to the pair of recombinant binding proteins and pharmaceutical compositions comprising said pair of recombinant binding proteins.

### CROSS-REFERENCE TO RELATED APPLICATIONS

This application is a continuation of International Application No. PCT/EP2023/081671, filed Nov. 14, 2023, which claims benefit of priority to European Application No. 22207437.9, filed Nov. 15, 2022, each of which is incorporated herein by reference in its entirety.

### SEQUENCE LISTING

The instant application contains a sequence listing which has been submitted electronically in .xml format and is hereby incorporated by reference in its entirety. Said .xml copy, created on May 9, 2025, is named P37841-US_Sequence_Listing and is 62,598 bytes in size.

### FIELD OF THE INVENTION

The present invention relates to a pair of recombinant binding proteins and its uses, e.g. for activation of an effector domain upon binding to a target cell. Specifically, the invention relates to the pair of recombinant binding proteins and pharmaceutical compositions comprising said pair of recombinant binding proteins.

### BACKGROUND OF THE INVENTION

Cancer treatment by bispecific antibodies targeting antigens expressed on the surface of cancer and T cells, e.g. via CD3, and thereby mediating ADCC towards the cancer cells provide dosing challenges due to off-target T cell activation, which is undesired.

EP3180361 discloses a pair of precursor molecules, wherein a binding site specifically binding to CD3 is activated on a target cell. Such precursor molecules comprise a Fab fragment, wherein to the C-terminus said Fab fragment a CH2 domain and a variable antibody domain, e.g. binding to CD3, is fused. Upon target cell binding of two precursor molecules comprising different variable domains, a functional antigen binding site (e.g. binding to CD3) is formed by association of said variable domains.

EP2802607 also discloses a pair of precursor molecules, wherein a binding site specifically binding to CD3 is activated on a target cell. These precursor molecules comprise a single chain Fv fragment capable of binding to a target cell and an antibody variable domain that associates with the complementary variable domain comprised in the other precursor molecule in order to form a functional CD3 binding site.

Yet there is still a need for alternative solutions to activate effector domains on a target site.

### SUMMARY OF THE INVENTION

The present invention relates to a pair of recombinant binding proteins, comprising (a) a first fusion polypeptide comprising (i) a first antigen binding domain capable of binding to a target antigen, (ii) a first part of an effector domain, and (iii) a first complementing domain capable of association with the first part of the effector domain, wherein the first part of the effector domain and the first complementing domain are connected via a peptide linker; and (b) a second fusion polypeptide comprising (i) a second antigen binding domain capable of binding to a target antigen, (ii) a second part of an effector domain, and (iii) a second complementing domain capable of association with the second part of the effector domain, wherein the first part of the effector domain and the first complementing domain are connected via a peptide linker; wherein the first and the second part of the effector domain are capable of associating with each other to form a functional effector domain, characterized in that the effector domain is an antibody Fab fragment.

One embodiment of the invention relates to the pair of recombinant binding proteins, wherein the first part of the effector domain comprises a variable heavy chain domain having a Q39E mutation, and wherein the first complementing domain comprises a variable light chain domain having a Q38E mutation, wherein the second part of the effector domain comprises a variable light chain domain having a Q38K mutation, and wherein the second complementing domain comprises a variable heavy chain domain having a Q39K mutation.

One embodiment of the invention relates to the pair of recombinant bonding proteins, wherein the first part of the effector domain and the first complementing domain are comprised in a single chain Fab fragment, and wherein the second part of the effector domain and the second complementing domain are comprised in a single chain Fab fragment, and wherein in the single chain Fab fragment of the first fusion polypeptide the VH and VL domain are exchanged with each other and wherein in the single chain Fab fragment of the second fusion polypeptide the CH1 and CL domains are exchanged with each other.

One embodiment of the invention relates to the pair of recombinant bonding proteins, wherein the first fusion polypeptide comprises a (heterodimeric) Fc domain, and wherein the second fusion polypeptide comprises a (heterodimeric) Fc domain, in one embodiment wherein the first fusion polypeptide as well as the second fusion polypeptide comprise a heterodimeric Fc domain comprising two CH3 domains, wherein one of the CH3 domains comprises the mutations S354C and T366W, and the other CH3 domain comprises the mutations Y349C, L368A and Y407V.

One embodiment of the invention relates to the pair of recombinant bonding proteins, wherein the effector domain is an anti-CD3 antibody binding domain.

Another aspect of the invention is a method for forming a functional effector domain from a pair of recombinant binding proteins according to one of the preceding claims, wherein the first antigen binding domain and the second antigen binding domain of the recombinant binding domains specifically bind to an epitope on a surface of a target cell, the method comprising contacting the pair of recombinant binding proteins with a target cell allowing the binding of the first fusion polypeptide and the second fusion polypeptide to the target cell.

Another aspect of the invention is a pharmaceutical formulation comprising the pair of recombinant binding proteins according to any one of the preceding claims and a pharmaceutically acceptable carrier.

According to the invention a functional effector domain comprised of a first part that is comprised in a first fusion polypeptide and a second part that is comprised in a second fusion polypeptide is formed by association of the first part and the second part upon binding of the first and the second fusion polypeptide on a target cell. Therapeutic application of pairs of recombinant binding proteins of the invention allows formation of an effector domain, e.g. an anti-CD3 binding site, at the target site only thus reducing undesired off target toxicity. Methods and pairs of recombinant binding proteins of the invention may be advantageously used for providing antigen binding proteins for therapeutic use, e.g., for the treatment of cancer.

### DESCRIPTION OF THE FIGURES

FIG. 1: Design and modular composition of inactive precursor modules in which the prodrug entity is covalently fused in a single-chain-Fab-like manner to yield a Fab.

FIG. 2: Generation of bispecific antibodies from monospecific precursors

FIG. 3: Generation of trispecific antibodies from monospecific precursors

FIGS. 4A-4D: FIG. 4A, Expression profile of the tumor antigens EGFR and HER2 on SK-BR-3 cells. FIG. 4B, Activation of CD3 binding functionality by previously inactive precursor molecules targeting HER2 on SK-BR-3 cells detected by T cell reporter assay. FIG. 4C, EGFR and HER2 expression on A431 cells. FIG. 4D, Activation of CD3 binding functionality by previously inactive precursor molecules targeting EGFR on A-431 cells detected by T cell reporter assay.

FIG. 5: Activation of CD3 binding functionality by previously inactive precursor molecules targeting (i) HER2 and (ii) EGFR on dual positive SK-BR-3 cells detected by T cell reporter assay.

FIG. 6: Generation of bispecific antibodies from one monospecific precursor and one non-specific precursor with covalently attached exchange units as a measure for in-solution exchange.

FIGS. 7A-7B: FIG. 7A, Activation of CD3 binding functionality by previously inactive precursor molecules (i) targeting HER2 and (ii) without targeting, harboring non-covalently attached exchange units on HER2 positive SK-BR-3 cells detected by T cell reporter assay as a measure of in-solution shuffling. FIG. 7B, Activation of CD3 binding functionality by previously inactive precursor molecules (i) targeting HER2 and (ii) without targeting, harboring covalently attached exchange units on HER2 positive SK-BR-3 cells detected by T cell reporter assay as a measure of in-solution shuffling.

FIG. 8: Generation of trispecific antibodies from monospecific precursors

FIG. 9: Activation of CD3 binding functionality by previously inactive precursor molecules targeting HER2 containing CD3 VH/VL in combination with Dig or non-binding “Nada” VH/VL in inner and outer positions on dual positive SK-BR-3 cells detected by T cell reporter assay. Nomenclature of the molecules: <Target> (Inner binder) (Outer binder) [Prodrug A/B]

FIG. 10: Activation of Dig binding functionality by previously inactive precursor molecules targeting HER2 containing CD3 VH/VL in combination with Dig or non-binding “Nada” VH/VL in inner and outer positions on Sk-Br-3 cells as detected by flow cytometry. Precursor molecules containing Dig and CD3 VH/VL pairs in different positions (inner or outer position) were added to Sk-Br-3 cells in a 96 well plate, either in isolation or in combination. After incubation and washing, digoxigenylated Cy5 dye (Dig-Cy5)

FIGS. 11A-11B: FIG. 11A, CD28 co-stimulation of T cells with HER2-targeted precursor molecules. HER2-positive Sk-Br-3 cells (60,000 cells per well) were co-cultured with Jurkat IL-2 promoter cells (100,000 cells per well) and were treated with increasing concentrations of Fab-PACE prodrug combinations. After incubation for 16 h at 37° C., ONE-Glo™ solution (Promega, Catalog No. E6120) was added to the plates and luminescence was measured according to the manufacturer's instructions. CD28 co-stimulation of Jurkat T cells was observed with CD28 in the inner as well as the outer position, indicated by the increase in luminescence of CD28-containing precursor molecule combinations compared to Nada-containing combinations. FIG. 11B, CD28 co-stimulation of Jurkat IL-2 promoter cells was monitored in the presence of individual HER2-targeted precursor molecules, as in FIG. 11A. No T cell activation or co-stimulation was observed with any of the individual prodrugs, even at high concentrations. Nomenclature of the Fab-PACE molecules in FIGS. 11A-11B is: <Target> (Inner binder) (Outer binder) [Prodrug A/B].

FIGS. 12A-12B: FIG. 12A, Generation of tetraspecific antibodies from monospecific precursors; FIG. 12B, Schematic representation of assay setup of the HEK-Blue IL-2 transactivation assay as described in Example 8

FIG. 13: IL-2 transactivation as described in Example 8. (A) Conversion of inactive PD-1-targeted precursor molecules into active IL-2 receptor agonists upon accumulation on PD-1-expressing cells. (B) Control experiment on PD-1 negative cells indicates that the activation of the active IL2 receptor agonists is target specific.

### DETAILED DESCRIPTION OF THE INVENTION

### 1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

The terms “a”, “an” and “the” generally include plural referents, unless the context clearly indicates otherwise.

Unless otherwise defined herein the term “comprising of” shall include the term “consisting of”.

The provision of two alternatives using the terms “either . . . or” designates mutually exclusive alternatives, unless the context clearly indicates otherwise.

The term “antigen binding domain” as used herein refers to a domain that specifically binds to a target antigen. The term includes antibody binding sites as well as other natural (e.g. receptors, ligands) or synthetic (e.g., DARPins) molecules capable of specifically binding to a target antigen.

The term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “binding site” or “antigen-binding site” as used herein denotes the region or regions of an antigen binding moiety to which the antigen actually binds. In case the antigen binding moiety is an antibody, the antigen-binding site includes antibody heavy chain variable domains (VH) and/or antibody light chain variable domains (VL), or pairs of VH/VL. Antigen-binding sites derived from antibodies that specifically bind to a target antigen can be derived a) from known antibodies specifically binding to the antigen or b) from new antibodies or antibody fragments obtained by de novo immunization methods using inter alia either the antigen protein or nucleic acid or fragments thereof or by phage display methods.

When being derived from an antibody, an antigen-binding site of an antibody according to the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding.

The term “valent” as used herein denotes the presence of a specified number of binding sites in an antibody molecule. A natural antibody, for example, has two binding sites and is bivalent. As such, the term “trivalent” denotes the presence of three binding sites in an antibody molecule.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv, scFab); and multispecific antibodies formed from antibody fragments.

“Specificity” refers to selective recognition of a particular epitope of an antigen by the antigen binding moiety, e.g. an antibody. Natural antibodies, for example, are monospecific. The term “monospecific antibody” as used herein denotes an antibody that has one or more binding sites each of which bind to the same epitope of the same antigen. “Multispecific antibodies” bind two or more different epitopes (for example, two, three, four, or more different epitopes). The epitopes may be on the same or different antigens. An example of a multispecific antibody is a “bispecific antibody” which binds two different epitopes. When an antibody possesses more than one specificity, the recognized epitopes may be associated with a single antigen or with more than one antigen.

An epitope is a region of an antigen that is bound by an antigen binding moiety, e.g. an antibody. The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three-dimensional structural characteristics, and/or specific charge characteristics.

As used herein, the terms “binding” and “specific binding” refer to the binding of the antibody or antigen binding moiety to an epitope of the antigen in an in vitro assay, preferably in a plasmon resonance assay (BIAcore®, GE-Healthcare Uppsala, Sweden) with purified wild-type antigen. In certain embodiments, an antibody or antigen binding moiety is said to specifically bind an antigen when it preferentially recognizes its target antigen in a complex mixture of proteins and/or macromolecules.

The affinity of the binding of an antibody to an antigen is defined by the terms kₐ (rate constant for the association of the antibody from the antibody/antigen complex), k_{D} (dissociation constant), and K_{D} (k_{D}/ka). In one embodiment binding or that/which specifically binds to means a binding affinity (K_{D}) of 10⁻⁸ mol/l or less, in one embodiment 10⁻⁸ M to 10⁻¹³ mol/l. Thus, an antigen binding moiety, particularly an antibody binding site, specifically binds to each antigen for which it is specific with a binding affinity (K_{D}) of 10⁻⁸ mol/l or less, e.g. with a binding affinity (K_{D}) of 10⁻⁸ to 10⁻¹³ mol/l. in one embodiment with a binding affinity (K_{D}) of 10⁻⁹ to 10⁻¹³ mol/l.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “constant domains” or “constant region” as used within the current application denotes the sum of the domains of an antibody other than the variable region. The constant region is not directly involved in binding an antigen but exhibits various effector functions.

Depending on the amino acid sequence of the constant region of their heavy chains, antibodies are divided in the “classes”: IgA, IgD, IgE, IgG and IgM, and several of these may are further divided into subclasses, such as IgG1, IgG2, IgG3, and IgG4, IgA1 and IgA2. The heavy chain constant regions that correspond to the different classes of antibodies are called α, δ, ε, γ and μ, respectively. The light chain constant regions (CL) which can be found in all five antibody classes are called κ (kappa) and λ (lambda).

The “constant domains” as used herein are, preferably, from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, MD (1991).

In wild type antibodies, the “hinge region” is a flexible amino acid stretch in the central part of the heavy chains of the IgG and IgA immunoglobulin classes, which links the two heavy chains by disulfide bonds, i.e. “interchain disulfide bonds” as they are formed between the two heavy chains. The hinge region of human IgG1 is generally defined as stretching from about Glu216, or about Cys226, to about Pro230 of human IgG1 (Burton, Molec. Immunol. 22:161-206 (1985)). By deleting cysteine residues in the hinge region or by substituting cysteine residues in the hinge region by other amino acids, such as serine, disulfide bond formation in the hinge region is avoided.

The “light chains” of antibodies from any vertebrate species can be assigned to one of two distinct types, called kappa (κ) and lambda (λ), based on the amino acid sequences of their constant domains. A wild type light chain typically contains two immunoglobulin domains, usually one variable domain (VL) that is important for binding to an antigen and a constant domain (CL).

Several different types of “heavy chains” exist that define the class or isotype of an antibody. A wild type heavy chain contains a series of immunoglobulin domains, usually with one variable domain (VH) that is important for binding antigen and several constant domains (CH1, CH2, CH3, etc.).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “CH2 domain” of a human IgG Fc region usually extends from an amino acid residue at about position 231 to an amino acid residue at about position 340. The multispecific antibody is devoid of a CH2 domain. By “devoid of a CH2 domain” is meant that the antibodies according to the invention do not comprise a CH2 domain.

The “CH3 domain” comprises the stretch of residues C-terminal to a CH2 domain in an Fc region (i.e. from an amino acid residue at about position 341 to an amino acid residue at about position 447 of an IgG). The “CH3 domains” herein are variant CH3 domains, wherein the amino acid sequence of the natural CH3 domain was subjected to at least one distinct amino acid substitution (i.e. modification of the amino acid sequence of the CH3 domain) in order to promote heterodimerization of the two CH3 domains facing each other within the multispecific antibody.

Typically, in the heterodimerization approaches known in the art, the CH3 domain of one heavy chain and the CH3 domain of the other heavy chain are both engineered in a complementary manner so that the heavy chain comprising one engineered CH3 domain can no longer homodimerize with another heavy chain of the same structure. Thereby the heavy chain comprising one engineered CH3 domain is forced to heterodimerize with the other heavy chain comprising the CH3 domain, which is engineered in a complementary manner.

One heterodimerization approach known in the art is the so-called “knobs-into-holes” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, which are herein included by reference. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

In combination with the substitutions according to the knobs-into-holes technology, additional interchain disulfide bonds may be introduced into the CH3 domains to further stabilize the heterodimerized polypeptides (Merchant, A. M., et al., Nature Biotech. 16 (1998) 677-681). Such interchain disulfide bonds are formed, e.g. by introducing the following amino acid substitutions into the CH3 domains: D399C in one CH3 domain and K392C in the other CH3 domain; Y349C in one CH3 domain and S354C in the other CH3 domain; Y349C in one CH3 domain and E356C in the other CH3 domain; Y349C in one CH3 domain and E357C in the other CH3 domain; L351C in one CH3 domain and S354C in the other CH3 domain; T394C in one CH3 domain and V397C in the other CH3 domain. A “cysteine mutation” as used herein refers to one amino acid substitution of an amino acid in a CH3 domain by cysteine that is capable of forming an interchain disulfide bond with another, matching, amino acid substitution of an amino acid in a second CH3 domain by cysteine.

Further techniques, apart from the “knobs-into-holes” technology as mentioned before, for modifying the CH3 domains in order to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knobs-into-holes technology” for the polypeptides provided by the invention. All those technologies involve engineering of CH3 domains in a complementary manner, by introduction of amino acids of opposite charge or different side chain volume, thereby supporting heterodimerization.

The term “polypeptide chain” as used herein refers to a linear organic polymer comprising a large number of amino acids linked together via peptide bonds. One or more polypeptide chains form a “polypeptide” or “protein”, wherein both terms are used interchangeably herein. Heterodimeric precursor polypeptides as provided in a set according to the invention comprise at least two polypeptide chains comprising a CH3 domain. Thus, a first polypeptide chain comprising a first CH3 domain is “associated” with a second polypeptide chain comprising a second CH3 domain to form a dimeric polypeptide. As the first CH3 domain and the second CH3 domain comprise amino acid substitutions according to the knobs-into-holes technology, the two polypeptide chains form a “heterodimer”, i.e. a dimer formed by two non-identical polypeptides.

Polypeptide chains may comprise one or more polypeptide domains. When the order of the polypeptide domains is indicated herein, it is indicated in N- to C-terminal direction.

Each one of the first and second fusion polypeptide comprises at least two polypeptide chains comprising a CH3 domain.

heavy chain/light chain vs heavy chain polypeptide/light chain polypeptide

An “isolated” polypeptide, e.g. an antibody, is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991). In particular, for variable domains and for the light chain constant domain CL of kappa and lambda isotype, the Kabat numbering system (see pages 647-660) of Kabat, et al., Sequences of Proteins of Immunological Interest, 5th ed., Public Health Service, National Institutes of Health, Bethesda, MD (1991) is used and for the constant heavy chain domains (CH1, Hinge, CH2 and CH3) the Kabat EU index numbering system (see pages 661-723) is used. Amino acid positions provided herein are usually indicated by

Amino acid “substitutions” or “replacements” or “mutations” (all terms are herein used interchangeably) within the polypeptide chains are prepared by introducing appropriate nucleotide changes into the antibody DNA, or by nucleotide synthesis. Such modifications can be performed, however, only in a very limited range. For example, the modifications do not alter the above-mentioned antibody characteristics such as the IgG isotype and antigen binding, but may further improve the yield of the recombinant production, protein stability or facilitate the purification. In certain embodiments, antibody variants having one or more conservative amino acid substitutions are provided. A “double mutation” as referred herein means that both of the indicated amino acid substitutions are present in the respective polypeptide chain.

The term “amino acid” as used herein denotes an organic molecule possessing an amino moiety located at α-position to a carboxylic group. Examples of amino acids include: arginine, glycine, ornithine, lysine, histidine, glutamic acid, asparagic acid, isoleucine, leucine, alanine, phenylalanine, tyrosine, tryptophane, methionine, serine, proline. The amino acid employed is optionally in each case the L-form. The term “positively charged” or “negatively charged” amino acid refers to the amino acid side-chain charge at pH 7.4. Amino acids may be grouped according to common side-chain properties:
(1) hydrophobic: Norleucine, Met, Ala, Val, Leu, Ile, Trp, Tyr, Phe;
(2) neutral hydrophilic: Cys, Ser, Thr, Asn, Gln;
(3) acidic or negatively charged: Asp, Glu;
(4) basic or positively charged: His, Lys, Arg;
(5) residues that influence chain orientation: Gly, Pro.

TABLE A
Amino acids with specific properties

| | | | Side-chain | Side-chain |
|---|---|---|---|---|
| Amino Acid | 3-Letter | 1-Letter | polarity | charge (pH 7.4) |
| Alanine | Ala | A | nonpolar | neutral |
| Arginine | Arg | R | basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | basic polar | positive (10%) |
| | | | | neutral (90%) |
| Isoleucine | Ile | I | nonpolar | neutral |
| Leucine | Leu | L | nonpolar | neutral |
| Lysine | Lys | K | basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

The term “purified” as used herein refers to polypeptides, that are removed from their natural environment or from a source of recombinant production, or otherwise isolated or separated, and are at least 60%, e.g., at least 80%, free from other components, e.g. membranes and microsomes, with which they are naturally associated. Purification of antibodies (recovering the antibodies from the host cell culture) is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. with affinity media for the purification of kappa or lambda-isotype constant light chain domains, e.g. KappaSelect or LambdaSelect), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretic methods (such as gel electrophoresis, capillary electrophoresis) (Vijayalakshmi, M. A., Appl. Biochem. Biotech. 75 (1998) 93-102).

The term “peptide linker” as used herein denotes a peptide with amino acid sequences, which is preferably of synthetic origin. Within fusion polypeptides as used for the invention, peptide linkers may be used for fusing additional polypeptide domains, like antibody fragments, to the C- or N-terminus of an individual polypeptide chain. In one embodiment said peptide linkers are peptides with an amino acid sequence with a length of at least 5 amino acids, in another embodiment with a length of 5 to 100 amino acids, in yet another embodiment of 10 to 50 amino acids. In one embodiment the peptide linker is a glycine-serine linker. In one embodiment the peptide linker is a peptide consisting of glycine and serine amino acid residues. In one embodiment said peptide linker is

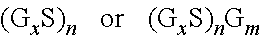

with G=glycine, S=serine, and
x=3, n=3, 4, 5 or 6, m=0, 1, 2 or 3; or
x=4, n=2, 3, 4 or 5, m=0, 1, 2 or 3.

In one embodiment x=4 and n=2 or 3, in another embodiment x=4, n=2. In one embodiment said peptide linker is (G₄S)₂.

The term “valent” as used herein denotes the presence of a specified number of binding sites in an antigen binding molecule. A natural antibody for example has two binding sites and is bivalent. As such, the term “trivalent” denotes the presence of three binding sites in an antigen binding molecule.

Polypeptides according to the invention are produced by recombinant means. Methods for recombinant production of polypeptides, e.g. antibodies, are widely known in the state of the art and comprise protein expression in prokaryotic and eukaryotic host cells with subsequent isolation of the polypeptide and usually purification to a pharmaceutically acceptable purity. For the expression of the polypeptides as aforementioned in a host cell, nucleic acids encoding the respective polypeptide chains are inserted into expression vectors by standard methods. Expression is performed in appropriate prokaryotic or eukaryotic host cells, like CHO cells, NS0 cells, SP2/0 cells, HEK293 cells, COS cells, PER. C6 cells, yeast, or E. coli cells, and the polypeptide is recovered from the cells (supernatant or cells after lysis). General methods for recombinant production of polypeptides, e.g. antibodies, are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

Polypeptides produced by host cells may undergo post-translational cleavage of one or more, particularly one or two, amino acids from the C-terminus of the polypeptide chain comprising a CH3 domain at the C-terminal end. Therefore, a polypeptide produced by a host cell by expression of a specific nucleic acid molecule encoding such polypeptide chain may include the full-length polypeptide chain including the full length CH3 domain, or it may include a cleaved variant of the full-length polypeptide chain (also referred to herein as a cleaved variant polypeptide chain). This may be the case where the final two C-terminal amino acids of the heavy chain are glycine (G446) and lysine (K447).

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a heterodimeric polypeptide” refers to one or more nucleic acid molecules encoding one or more polypeptide chains (or fragments thereof) of said heterodimeric polypeptide, including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a vector are capable of directing the expression of nucleic acids to which they are operatively linked. When the expression vector is introduced into an appropriate host cell, it can be transcribed and translated into a polypeptide. When transforming host cells in methods according to the invention, “expression vectors” are used; thereby the term “vector” in connection with transformation of host cells as described herein means “expression vector”. An “expression system” usually refers to a suitable host cell comprised of an expression vector that can function to yield a desired expression product.

As used herein, “expression” refers to the process by which a nucleic acid is transcribed into mRNA and/or to the process by which the transcribed mRNA (also referred to as a transcript) is subsequently translated into a peptide or polypeptide. The transcripts and the encoded polypeptides are individually or collectively referred to as gene products. If a nucleic acid is derived from genomic DNA, expression in a eukaryotic cell may include splicing of the corresponding mRNA.

The term “transformation” as used herein refers to process of transfer of a vector or a nucleic acid into a host cell. If cells without formidable cell wall barriers are used as host cells, transfection is carried out e.g. by the calcium phosphate precipitation method as described by Graham and Van der Eh, Virology 52 (1978) 546ff. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, F. N, et al., PNAS 69 (1972) 7110 et seq.

The term “host cell” as used in the current application denotes any kind of cellular system which can be engineered to generate the polypeptides provided with the invention.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., J. Immunol. Methods 194 (1996) 191-199.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer an antibody according to the invention by certain routes of administration, it may be necessary to coat the antibody with, or co-administer the antibody with, a material to prevent its inactivation. For example, the heterodimeric polypeptide may be administered to a subject in an appropriate carrier, for example, liposomes, or a diluent. Pharmaceutically acceptable diluents include saline and aqueous buffer solutions.

A pharmaceutical composition comprises an effective amount of the fusion polypeptides provided with the invention. An “effective amount” of an agent, e.g., a fusion polypeptide, refers to an amount effective, at dosages and for periods of time necessary, to achieve the desired therapeutic or prophylactic result. In particular, the “effective amount” denotes an amount of a heterodimeric polypeptide of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the heterodimeric polypeptide molecules used, disease state being treated, the severity or the disease treated, the age and relative health of the subject, the route and form of administration, the judgment of the attending medical or veterinary practitioner, and other factors.

A “pharmaceutically acceptable carrier” refers to an ingredient in a pharmaceutical formulation, other than an active ingredient, which is nontoxic to a subject. Pharmaceutically acceptable carriers include any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, in one embodiment the carrier is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### 2. Detailed Description of the Embodiments of the Invention

The invention provides a pair of fusion polypeptides applicable, e.g., for in vivo generation of a functional effector domain. One application is the on-cell generation of an antigen binding site by association of a newly formed antigen binding site, e.g. an anti-CD3 binding site. The pair of recombinant fusion polypeptides may also be referred as a pair of precursor polypeptides, as they are capable of forming a polypeptide complex with each other once the parts of the effector domain that are comprised in the two fusion polypeptides associate with each other and form the functional effector domain.

The pair of recombinant fusion proteins comprises a first fusion polypeptide comprising a part of an effector domain that is complemented with a complementing domain. When associated with each other, the part of the effector domain and the complementing domain form a non-functional antigen binding domain. The second fusion polypeptide comprises a second part of the effector domain that is complemented with a second complementing domain. When associated with each other, the second part of the effector domain and the complementing domain also form a non-functional antigen binding domain. According to the invention, the effector domain is an antibody Fab fragment, wherein the first part of the effector domain is an antibody light chain polypeptide, wherein the second part of the effector domain is an antibody heavy chain polypeptide (CH1-VH).

When both, the first and the second fusion polypeptide, are brought into close proximity with each other, the complemented parts of the effector domains dissociate from the respective complementing domains and associate with each other, forming a functional antigen binding site. This is supported by pairs of mutations with charged amino acids in the interface of the respective part of the effector domain and the complementing domain. While in the first fusion polypeptide the interface of the first part of the effector domain and the first complementing domain comprises at least one pair of amino acid mutations having a positive charge, in the second fusion polypeptide the interface of the second part of the effector domain and the second complementing domain comprises at least one pair of amino acid mutations having a negative charge.

### Pair of Polypeptides

In one aspect the invention relates to a pair of recombinant binding proteins, comprising (a) a first fusion polypeptide comprising (i) a first antigen binding domain capable of binding to a target antigen, (ii) a first part of an effector domain, and (iii) a first complementing domain capable of association with the first part of the effector domain, wherein the first part of the effector domain and the first complementing domain are connected via a peptide linker; and (b) a second fusion polypeptide comprising (i) a second antigen binding domain capable of binding to a target antigen, (ii) a second part of an effector domain, and (iii) a second complementing domain capable of association with the second part of the effector domain, wherein the first part of the effector domain and the first complementing domain are connected via a peptide linker; wherein the first and the second part of the effector domain are capable of associating with each other to form a functional effector domain, characterized in that the effector domain is an antibody Fab fragment.

### (A) Antigen Binding Domains

The first fusion polypeptide comprises a first antigen binding domain. The second fusion polypeptide comprises a second antigen binding domain. In one embodiment the antigen binding domain is an antibody-derived antigen binding domain. In one embodiment the antibody-derived antigen binding domain is an antibody fragment. In one embodiment the antibody fragment is a Fab fragment. In one embodiment the first fusion polypeptide and the second fusion polypeptide comprise an antibody-derived antigen binding domain.

In one embodiment the antigen binding domain comprised in the first fusion polypeptide and/or the second fusion polypeptide specifically binds to a target cell. In one embodiment the antigen binding domain specifically binds to an epitope on the surface of a target cell. In one embodiment the antigen binding domains of the first fusion polypeptide and the second fusion polypeptide bind to the same epitope on the surface of a target cell. In one embodiment the antigen binding domains of the first fusion polypeptide and the second fusion polypeptide bind to a different epitope on the surface of a target cell. In one embodiment the antigen binding domains of the first fusion polypeptide and the second fusion polypeptide bind to a different epitope of the same antigen on the surface of a target cell.

### (B) Effector Domain

The first fusion polypeptide comprises a first part of an effector domain. The second fusion polypeptide comprises a second part of an effector domain. The first and the second part of the effector domain are capable of associating with each other to form a functional effector domain.

In one embodiment the effector domain is dimer.

In one embodiment the effector domain is an antigen binding domain. In one embodiment the effector domain is an antibody-derived antigen binding domain. In one embodiment the effector domain is an antibody fragment. In one embodiment the effector domain is a Fab fragment. In one embodiment the effector domain is an antibody Fab fragment, wherein the first part of the effector domain is an antibody light chain polypeptide, wherein the second part of the effector domain is an antibody heavy chain polypeptide (CH1-VH).

In one embodiment the first part of the effector domain is an antibody heavy chain polypeptide and the first complementing domain is an antibody light chain polypeptide, and the second part of the effector domain is an antibody light chain polypeptide and the second complementing domain is an antibody heavy chain polypeptide.

In one embodiment the effector domain specifically binds to an antigen. In one embodiment the effector domain specifically binds to a T-cell antigen, particularly an activating T cell antigen. In one embodiment the effector domain specifically binds to CD3, particularly CD3E. In one embodiment the effector domain specifically binds to human CD3.

In one embodiment the effector domain specifically binds to human CD28.

In one embodiment the effector domain specifically binds to a cell surface receptor. In one embodiment the effector domain specifically binds to domains of a cytokine receptor. In one embodiment the effector domain specifically binds to IL2Rgamma. In one embodiment the effector domain specifically binds to IL2Rbeta.

The first fusion polypeptide comprises a first part of a first effector domain and a first part of a second effector domain. The second fusion polypeptide comprises a second part of a first effector domain and a second part of a second effector domain. The first and the second part of the effector domains are capable of associating with each other to form respective functional first and second effector domains.

In one embodiment the second effector domain specifically binds to a factor, preferably a cell surface factor, which additionally further activates the immune response at a tumor.

### (C) Complementing Domain

The first fusion polypeptide comprises a first complementing domain. The first complementing domain is capable of association with the first part of the effector domain. The second fusion polypeptide comprises a second complementing domain. The second complementing domain is capable of association with the second part of the effector domain.

In one embodiment, the first complementing domain is capable of association with the second complementing domain to form a dimer of the first and the second complementing domain (herein also referred to as a “complementing domain dimer”).

In one embodiment the first complementing domain and the second complementing domain form a non-functional antigen binding domain, when the first and the second complementing domain are associated with each other. In one embodiment the first complementing domain and the second complementing domain form a functional antigen binding domain, when the first and the second complementing domain are associated with each other.

In one embodiment the complementing domain dimer is an antigen binding domain. In one embodiment the complementing domain dimer is an antibody-derived antigen binding domain. In one embodiment the complementing domain dimer is an antibody fragment. In one embodiment the effector domain is a Fab fragment.

### (D) Domain Arrangement and Amino Acid Substitutions

The first fusion polypeptide and the second fusion polypeptides are precursor polypeptides that are capable of forming a complex with each other once the first part of the effector domain and the second part of the effector domain associate with each other. In order to support this dimerization, the first fusion polypeptide and the second fusion polypeptide are arranged such that the association of the two parts of the effector domains is preferred over an association of the individual parts of the effector domains with their respective complementing domain.

In one embodiment the first part of the effector domain is comprises an antibody variable domain and the first complementing domain comprises a complementary antibody variable domain. In one embodiment the first part of the effector domain comprises a VL domain and the first complementing domain comprises a VH domain. In one embodiment the first part of the effector domain comprises a VH domain and the first complementing domain comprises a VL domain. In one embodiment said VH and said VL domain comprise an interface with at least one amino acid substitution in each domain introducing an amino acid of positive charge. In one embodiment said VH and said VL domain comprise an interface with at least one amino acid substitution in each domain introducing an amino acid of negative charge. In one embodiment the VH domain comprises a Q39E mutation and the VL domain comprises a Q38E mutation.

In one embodiment the second part of the effector domain is comprises an antibody variable domain and the second complementing domain comprises a complementary antibody variable domain. In one embodiment the second part of the effector domain comprises a VL domain and the second complementing domain comprises a VH domain. In one embodiment the second part of the effector domain comprises a VH domain and the second complementing domain comprises a VL domain. In one embodiment said VH and said VL domain comprise an interface with at least one amino acid substitution in each domain introducing an amino acid of positive charge. In one embodiment said VH and said VL domain comprise an interface with at least one amino acid substitution in each domain introducing an amino acid of negative charge. In one embodiment the VH domain comprises a Q39K mutation and the VL domain comprises a Q38K mutation.

In one embodiment the first fusion polypeptide comprises a first part of an effector domain and a first complementing domain comprising a pair of a VL domain and a VH domain, wherein the the VH domain comprises a Q39E mutation and the VL domain comprises a Q38E mutation. In one embodiment the first fusion polypeptide comprises a first part of an effector domain comprising a VH domain comprising a Q39E mutation and a first complementing domain comprising a VL domain comprising a Q38E mutation.

In one embodiment the second fusion polypeptide comprises a second part of an effector domain and a second complementing domain comprising a pair of a VL domain and a VH domain, wherein the the VH domain comprises a Q39K mutation and the VL domain comprises a Q38K mutation. In one embodiment the second fusion polypeptide comprises a second part of an effector domain comprising a VL domain comprising a Q38K mutation and a second complementing domain comprising a VH domain comprising a Q39K mutation.

In one embodiment the first fusion polypeptide comprises a first part of an effector domain comprising a VH domain comprising a Q39E mutation and a first complementing domain comprising a VL domain comprising a Q38E mutation, and the second fusion polypeptide comprises a second part of an effector domain comprising a VL domain comprising a Q38K mutation and a second complementing domain comprising a VH domain comprising a Q39K mutation. Due to the amino acid mutations with charged amino acids the interaction between the parts of the effector domain with their respective complementing domain is weaker (as the interface comprises amino acids of the same charge) while the interaction between both parts of the effector domain and, optionally, both complementing domains, is stronger as the interface comprises amino acids of the opposite charge.

In one embodiment the first part of the effector domain is an antibody light chain polypeptide. In one embodiment the first complementing domain is an antibody heavy chain polypeptide.

In one embodiment the second part of the effector domain is an antibody heavy chain polypeptide. In one embodiment the second complementing domain is an antibody light chain polypeptide.

In one embodiment, the the first part of the effector domain is an antibody light chain polypeptide, the first complementing domain is an antibody heavy chain polypeptide, the second part of the effector domain is an antibody heavy chain polypeptide, and the second complementing domain is an antibody light chain polypeptide.

In one embodiment wherein the first fusion polypeptide is a recombinant antibody consisting of three polypeptides: (i) an antibody light chain, and (ii) an antibody heavy chain, wherein the variable domains of said antibody light chain and said antibody heavy chain form the first antigen binding domain, and (iii) an antibody heavy chain/light chain fusion polypeptide, comprising an antibody light chain comprising the first part of the effector domain fused via a peptide linker to an antibody heavy chain comprising the first complementing domain, wherein the C-terminus of the antibody light chain is fused to the N-terminus of the antibody heavy chain; and wherein the second fusion polypeptide is a recombinant antibody consisting of three polypeptides: (i) an antibody light chain, and (ii) an antibody heavy chain, wherein the variable domains of said antibody light chain and said antibody heavy chain form the second antigen binding domain, and (iii) an antibody heavy chain/light chain fusion polypeptide, comprising an antibody light chain comprising the second part of the effector domain fused via a peptide linker to an antibody heavy chain comprising the second complementing domain, wherein the C-terminus of the antibody light chain is fused to the N-terminus of the antibody heavy chain.

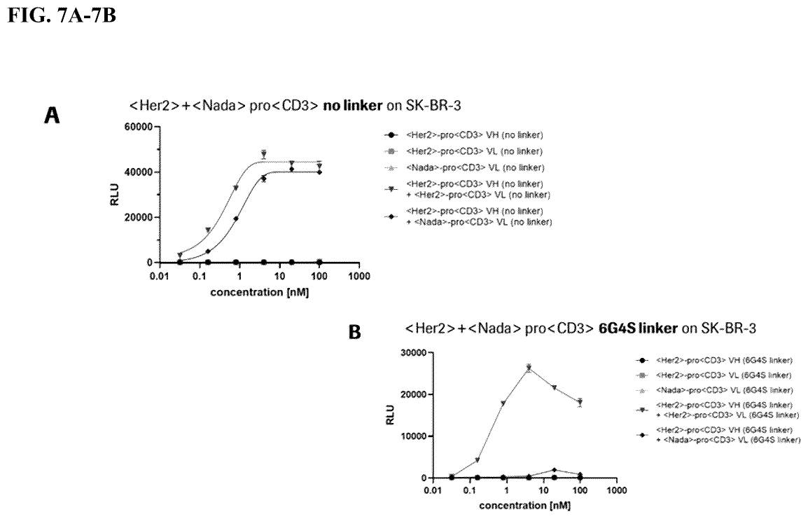

In one embodiment the the first part of the effector domain and the first complementing domain are comprised in a single chain Fab fragment. In one embodiment the the second part of the effector domain and the second complementing domain are comprised in a single chain Fab fragment. It was observed that using single chain Fab fragments prominently reduced remaining in solution activation of the effector domain (FIG. 7).

In one embodiment, the C-terminus of the first part of the effector domain is fused via a peptide linker to the N-terminus of the first complementing domain.

In one preferred embodiment of the invention, the linker is a peptide of at least 20 amino acids. In one preferred embodiment of the invention, the linker is a peptide of at least 25 amino acids. In another embodiment of the invention, the linker is a peptide of 25-70 amino acids. In another embodiment of the invention, the linker is a peptide of 25-35 amino acids. In one embodiment of the invention, the linker is a glycine-serine linker. In one embodiment of the invention, the linker is a peptide consisting of glycine and serine residues. In one embodiment of the invention, the glycine-serine linkers comprises at least more than 5, preferentially more than six, repeats of (Gly-Gly-Gly-Gly-Ser).

In one embodiment, wherein the first fusion polypeptide and the second fusion polypeptides comprise single chain Fab fragments comprising the parts of the effector domain and the respective complementing domain, the single chain Fab fragments comprise a domain crossover such that the first part of the effector domain is capable of association with the second part of the effector domain. Domain crossovers in multispecific antibodies are known in the art, e.g. the CrossMab-technology as disclosed in Schäfer et al (Schaefer W, Regula J T, Bähner M, Schanzer J, Croasdale R, Dürr H, Gassner C, Georges G, Kettenberger H, Imhof-Jung S, Schwaiger M, Stubenrauch K G, Sustmann C, Thomas M, Scheuer W, Klein C. Proc Natl Acad Sci USA. 2011 Jul. 5; 108 (27): 11187-92, Klein C, Sustmann C, Thomas M, Stubenrauch K, Croasdale R, Schanzer J, Brinkmann U, Kettenberger H, Regula J T, Schaefer W. MAbs. 2012 November-December; 4 (6): 653-63. doi: 10.4161/mabs.21379. Epub 2012 Aug. 27).

This allows recombinant expression of the first and the second fusion polypeptide with an improved side product profile. Furthermore, the use of different domain crossovers in the first and second fusion polypeptide allows association of the two parts of the effector domain with each other.

In one embodiment the single chain Fab fragment of the first fusion polypeptide comprises a domain crossover of the VH and the VL domains, i.e. the VH and the VL domain are exchanged with each other, and the single chain Fab fragment of the second fusion polypeptide comprises a domain crossover of the CH1 and CL domains, i.e. the CH1 and the CL domain are exchanged with each other.

In one embodiment the first fusion polypeptide comprises a first part of an effector domain comprising from N- to C-terminal direction a VH domain and a CL domain, and a first complementing domain comprising from N- to C-terminal direction a VL domain and a CH1 domain, and the second fusion polypeptide comprises a second part of an effector domain comprising from N- to C-terminal direction a VL domain and a CH1 domain, and a second complementing domain comprising from N- to C-terminal direction a VH domain and a CL domain.

In one embodiment the single chain Fab fragment of the first fusion polypeptide comprises from N- to C-terminal direction a VH domain, a CL domain, a peptide linker, a VL domain and a CH1 domain, and wherein the single chain Fab fragment of the second fusion polypeptide comprises from N- to C-terminal direction a VL domain, a CH1 domain, a peptide linker, a VH domain and a CL domain.

In one embodiment the first fusion polypeptide comprises a first part of an effector domain comprising from N- to C-terminal direction a VH domain comprising a Q39E mutation and a CL domain, and a first complementing domain comprising from N- to C-terminal direction a VL domain comprising a Q38E mutation and a CH1 domain, and the second fusion polypeptide comprises a second part of an effector domain comprising from N- to C-terminal direction a VL domain comprising a Q38K mutation and a CH1 domain, and a second complementing domain comprising from N- to C-terminal direction a VH domain comprising a Q39K mutation and a CL domain.

In one embodiment the single chain Fab fragment of the first fusion polypeptide comprises from N- to C-terminal direction a VH domain comprising a Q39E mutation, a CL domain, a peptide linker, a VL domain comprising a Q38E and a CH1 domain, and wherein the single chain Fab fragment of the second fusion polypeptide comprises from N- to C-terminal direction a VL domain comprising a Q38K mutation, a CH1 domain, a peptide linker, a VH domain comprising a Q39K mutation and a CL domain.

### (E) Fc Domains

In one embodiment the first fusion polypeptide comprises a Fc domain, particularly a heterodimeric Fc domain. In one embodiment the second fusion polypeptide comprises a Fc domain, particularly a heterodimeric Fc domain. In one embodiment, the first fusion polypeptide and the second fusion polypeptide comprise a, preferably heterodimeric, Fc domain.

In one embodiment the Fc domain comprises knob-into-hole mutations. In one embodiment the first fusion polypeptide as well as the second fusion polypeptide comprise a heterodimeric Fc domain comprising two CH3 domains, wherein one of the CH3 domains comprises the mutations S354C and T366W, and the other CH3 domain comprises the mutations Y349C, L368A and Y407V. This allows recombinant expression of the first and the second fusion polypeptide with an improved side product profile.

### (F) Antibody Isotypes

In one embodiment of the invention, the first and second fusion polypeptides comprise immunoglobulin constant regions of one or more immunoglobulin classes. Immunoglobulin classes include IgG, IgM, IgA, IgD, and IgE isotypes and, in the case of IgG and IgA, their subtypes. In one embodiment of the invention, the precursor polypeptide has a constant domain structure of an IgG type antibody.

In one embodiment of the invention the CH3 domains comprised in a first and second fusion polypeptide are of mammalian IgG class. In one embodiment of the invention the CH3 domains comprised in a first and second fusion polypeptide are of mammalian IgG1 subclass. In one embodiment of the invention the CH3 domains comprised in a first and second fusion polypeptide are of mammalian IgG4 subclass.

In one embodiment of the invention the CH3 domains comprised in a first and second fusion polypeptide are of human IgG class. In one embodiment of the invention the CH3 domains comprised in a first and second fusion polypeptide are of human IgG1 subclass. In one embodiment of the invention the CH3 domains comprised in a first and second fusion polypeptide are of human IgG4 subclass.

In one embodiment the constant domains of a first and second fusion polypeptide according to the invention are of human IgG class. In one embodiment the constant domains of a first and second fusion polypeptide according to the invention are of human IgG1 subclass. In one embodiment the constant domains of a first and second fusion polypeptide according to the invention are of human IgG4 subclass.

In one embodiment, the first and second fusion polypeptides are devoid of a CH4 domain.

In one embodiment of the invention the constant domains of the first and second fusion polypeptide according to the invention are of the same immunoglobulin subclass. In one embodiment of the invention the variable domains and constant domains of a first and second fusion polypeptide according to the invention are of the same immunoglobulin subclass.

In one embodiment of the invention the first and second fusion polypeptide is an isolated precursor polypeptide.

In one embodiment, a heterodimeric fusion polypeptide comprise a polypeptide chain including a CH3 domain includes a full length CH3 domain or a CH3 domain, wherein one or two C-terminal amino acid residues, i. e. G446 and/or K447 are not present.

### Method for Forming a Functional Effector Domain and Recombinant Methods

In another aspect the invention relates to a method or forming a functional effector domain from a pair of recombinant binding proteins according to the invention, wherein the first antigen binding domain and the second antigen binding domain of the recombinant binding proteins specifically bind to an epitope on a surface of a target cell, the method comprising contacting the pair of recombinant binding proteins with a target cell under conditions allowing the binding of the first fusion polypeptide and the second fusion polypeptide to the target cell.

Fusion polypeptides according to the invention are prepared by recombinant methods. Thus, the invention also relates to a method for the preparation of a fusion polypeptide according to the invention, comprising culturing a host cell comprising a nucleic acid encoding for the fusion polypeptide under conditions suitable for the expression of the fusion polypeptide.

In one aspect, a method of making a fusion polypeptide of the invention is provided, wherein the method comprises culturing a host cell comprising nucleic acid(s) encoding the fusion polypeptide, as provided above, under conditions suitable for expression of the fusion polypeptide, and optionally recovering the fusion polypeptide from the host cell (or host cell culture medium).

In one embodiment the method comprises the steps of transforming a host cell with expression vectors comprising nucleic acids encoding the fusion polypeptide, culturing said host cell under conditions that allow synthesis of said fusion polypeptide, and recovering said fusion polypeptide from said host cell culture.

For recombinant production of a fusion polypeptide, nucleic acids encoding the fusion polypeptide, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the polypeptide chains of the fusion polypeptide) or produced by recombinant methods or obtained by chemical synthesis.

Suitable host cells for cloning or expression of antibody-encoding vectors include prokaryotic or eukaryotic cells described herein. For example, fusion polypeptides may be produced in bacteria. For expression of polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the fusion polypeptide may be isolated from the bacterial cell paste in a soluble fraction and can be further purified.

In addition to prokaryotes, eukaryotic microbes such as filamentous fungi or yeast are suitable cloning or expression hosts for vectors encoding for fusion polypeptides of the invention, including fungi and yeast strains whose glycosylation pathways have been “humanized”, resulting in the production of a polypeptide with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) fusion polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese Hamster Ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

In one aspect the invention provides an isolated nucleic acid encoding for a fusion polypeptide of the invention. In one aspect the invention provides an expression vector comprising a nucleic acid according to the invention. In another aspect the invention provides a host cell comprising the nucleic acid of the invention.

In one embodiment the method of the invention includes the provision of the pair of recombinant binding proteins according to the invention. Therefore, the first fusion polypeptide and the second fusion polypeptide are expressed by recombinant means. In one embodiment the first fusion polypeptide and the second fusion polypeptide are expressed in eukaryotic cells, preferably HEK293 cells or CHO cells. In one embodiment the first fusion polypeptide and the second fusion polypeptide are purified after recombinant expression.

In one embodiment the first fusion protein and the second fusion protein are contacted with a target cell, in one embodiment in an aqueous solution. The first antigen binding domain and the second antigen binding domain bind to a target antigen on the target cell. As the binding of the two precursor proteins brings the parts of the effector domains into close proximity, the first and the second part of the effector domain associated with each other to form a functional effector domain. Optionally, the first and second complementing domain also associate with each other, optionally to form another functional domain, e.g. an antigen binding domain.

### Therapeutic Application

The pair of recombinant binding proteins may be used in therapy, e.g. cancer therapy.

Thus, one aspect of the invention is the pair of recombinant binding proteins for use as a medicament. In one embodiment the effector domain is a CD3 specifically binds to CD3 and the pair of recombinant binding proteins is for use as a medicament for the treatment of cancer.

Another aspect is a method method of treating an individual having a disease comprising administering to the individual an effective amount of the pair of recombinant binding proteins of the invention or the pharmaceutical composition of the invention.

### 3. Specific Embodiments of the Invention

In the following specific embodiments of the invention are listed.
1. A pair of recombinant binding proteins, comprising
   (a) a first fusion polypeptide comprising (i) a first antigen binding domain capable of binding to a target antigen, (ii) a first part of an effector domain, and (iii) a first complementing domain capable of association with the first part of the effector domain, wherein the first part of the effector domain and the first complementing domain are connected via a peptide linker; and
   (b) a second fusion polypeptide comprising (i) a second antigen binding domain capable of binding to a target antigen, (ii) a second part of an effector domain, and (iii) a second complementing domain capable of association with the second part of the effector domain, wherein the first part of the effector domain and the first complementing domain are connected via a peptide linker;
   wherein the first and the second part of the effector domain are capable of associating with each other to form a functional effector domain, characterized in that the effector domain is a Fab fragment.
2. The pair of recombinant binding proteins according to embodiment 1, wherein the antigen binding domain specifically binds to an epitope on the surface of a target cell.
3. The pair of recombinant binding proteins according to embodiment 2, wherein the antigen binding domains of the first fusion polypeptide and the second fusion polypeptide bind to the same epitope on the surface of a target cell.
4. The pair of recombinant binding proteins according to embodiment 2, wherein the antigen binding domains of the first fusion polypeptide and the second fusion polypeptide bind to different epitopes on the surface of a target cell.
5. The pair of recombinant binding proteins according to one of the previous embodiments, wherein the effector domain is an antigen binding domain.
6. The pair of recombinant binding proteins according to one of the previous embodiments, wherein the effector domain is an antibody heavy chain polypeptide and the first complementing domain is an antibody light chain polypeptide, and the second part of the effector domain is an antibody light chain polypeptide and the second complementing domain is an antibody heavy chain polypeptide.
7. The pair of recombinant binding proteins according to one of the previous embodiments, wherein the effector domain specifically binds to a T-cell antigen, particularly an activating T cell antigen.
8. The pair of recombinant binding proteins according to one of the previous embodiments, wherein the effector domain specifically binds to CD3.
9. The pair of recombinant binding proteins according to one of the previous embodiments, wherein the effector domain specifically binds to CD28.
10. The pair of recombinant binding proteins according to one of the previous embodiments, wherein the effector domain is an antibody Fab fragment, wherein the first part of the effector domain is an antibody light chain polypeptide, wherein the second part of the effector domain is an antibody heavy chain polypeptide (CH1-VH).
11. The pair of recombinant binding proteins according to one of the previous embodiments
wherein the first part of the effector domain is an antibody heavy chain polypeptide and the first complementing domain is an antibody light chain polypeptide, and
wherein the second part of the effector domain is an antibody light chain polypeptide and the second complementing domain is an antibody heavy chain polypeptide.
12. The pair of recombinant binding proteins according to one of the preceding embodiments, wherein the complementing domain dimer is an antibody-derived antigen binding domain.
13. The pair of recombinant binding proteins according to one of the preceding embodiments, wherein the first complementing domain and the second complementing domain form a non-functional antigen binding domain, when the first and the second complementing domain are associated with each other.
14. The pair of recombinant binding proteins according to one of the preceding embodiments, wherein the Fc domain comprises knob-into-hole mutations.
15. The pair of recombinant binding proteins according to one of the preceding embodiments, wherein the first part of the effector domain comprises a variable heavy chain domain having a Q39E mutation, and wherein the first complementing domain comprises a variable light chain domain having a Q38E mutation,
wherein the second part of the effector domain comprises a variable light chain domain having a Q38K mutation, and wherein the second complementing domain comprises a variable heavy chain domain having a Q39K mutation.
16. The pair of recombinant binding proteins according to one of the preceding embodiments, wherein the first part of the effector domain and the first complementing domain are comprised in a single chain Fab fragment.
17. The pair of recombinant binding proteins according to one of the preceding embodiments, wherein the second part of the effector domain and the second complementing domain are comprised in a single chain Fab fragment.
18. The pair of recombinant binding proteins according to one of embodiments 15 or 16, wherein in the single chain Fab fragment of the first fusion protein the VH and VL domain are exchanged with each other and wherein in the single chain Fab fragment of the second fusion protein the CH1 and CL domains are exchanged with each other.
19. The pair of recombinant binding proteins according to one of embodiments 15 or 16, wherein the single chain Fab fragment of the first fusion protein comprises from N- to C-terminal direction a VH domain, a CL domain, a peptide linker, a VL domain and a CH1 domain, and wherein the single chain Fab fragment of the second fusion protein comprises from N- to C-terminal direction a VL domain, a CH1 domain, a peptide linker, a VH domain and a CL domain.
20. The pair of recombinant binding proteins according to one of the preceding embodiments, wherein the first antigen binding domain is an antibody Fab fragment.
21. The pair of recombinant binding proteins according to one of the preceding embodiments, wherein the second antigen binding domain is an antibody Fab fragment.
22. The pair of recombinant binding proteins according to one of the preceding embodiments, wherein the first fusion polypeptide comprises a (heterodimeric) Fc domain.
23. The pair of recombinant binding proteins according to one of the preceding embodiments, wherein the second fusion polypeptide comprises a (heterodimeric) Fc domain.
24. The pair of recombinant binding proteins according to one of the preceding embodiments, wherein the first fusion polypeptide as well as the second fusion polypeptide comprise a heterodimeric Fc domain comprising two CH3 domains, wherein one of the CH3 domains comprises the mutations S354C and T366W, and the other CH3 domain comprises the mutations Y349C, L368A and Y407V.
25. The pair of recombinant binding proteins according to one of the preceding embodiments, wherein the first fusion polypeptide is a recombinant antibody consisting of three polypeptides: (i) an antibody light chain, and (ii) an antibody heavy chain, wherein the variable domains of said antibody light chain and said antibody heavy chain form the first antigen binding domain, and (iii) an antibody heavy chain/light chain fusion polypeptide, comprising an antibody light chain comprising the first part of the effector domain fused via a peptide linker to an antibody heavy chain comprising the first complementing domain, wherein the C-terminus of the antibody light chain is fused to the N-terminus of the antibody heavy chain; and
wherein the second fusion polypeptide is a recombinant antibody consisting of three polypeptides: (i) an antibody light chain, and (ii) an antibody heavy chain, wherein the variable domains of said antibody light chain and said antibody heavy chain form the second antigen binding domain, and (iii) an antibody heavy chain/light chain fusion polypeptide, comprising an antibody light chain comprising the second part of the effector domain fused via a peptide linker to an antibody heavy chain comprising the second complementing domain, wherein the C-terminus of the antibody light chain is fused to the N-terminus of the antibody heavy chain.
26. The pair of recombinant binding proteins, wherein the effector domain is an antibody binding domain.
27. The pair of recombinant binding proteins, wherein the effector domain is an anti-CD3 antibody binding domain.
28. A method for forming a functional effector domain from a pair of recombinant binding proteins according to one of the preceding embodiments, wherein the first antigen binding domain and the second antigen binding domain of the recombinant binding proteins specifically bind to an epitope on a surface of a target cell, the method comprising contacting the pair of recombinant binding proteins with a target cell under conditions allowing the binding of the first fusion polypeptide and the second fusion polypeptide to the target cell.
29. A pharmaceutical formulation comprising the pair of recombinant binding proteins according to any one of the preceding embodiments and a pharmaceutically acceptable carrier.

TABLE B
Amino acid sequences

| | |
|---|---|
| SEQ ID NO: 1 | <Her2>pro<CD3>VH, <Her2>pro<CD3>VL, <Her2>pro<CD3> VH (no linker), |
| | <Her2>pro<CD3> VL (no linker) |
| | Her2 light chain |
| | DIQMTQSPSSLSASVGDRVTITCRASQDVNTAVAWYQQKPGKAPKLLIYSASFLYSGVPSRFSGSR |
| | SGTDFTLTISSLQPEDFATYYCQQHYTTPPTFGQGTKVEIKRTVAAPSVFIFPPSDEQLKSGTASV |
| | VCLLNNFYPREAKVQWKVDNALQSGNSQESVTEQDSKDSTYSLSSTLTLSKADYEKHKVYACEVTH |
| | QGLSSPVTKSFNRGEC |
| SEQ ID NO: 2 | <Her2>pro<CD3>VH, <Her2>pro<CD3>VL, <Her2>pro<CD3> VH (no linker), |
| | <Her2>pro<CD3> VL (no linker) |
| | Her2 heavy chain |
| | EVQLVESGGGLVQPGGSLRLSCAASGFNIKDTYIHWVRQAPGKGLEWVARIYPTNGYTRYADSVKG |
| | RFTISADTSKNTAYLQMNSLRAEDTAVYYCSRWGGDGFYAMDYWGQGTLVTVSSASTKGPSVFPLA |
| | PSSKSTSGGTAALGCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPEAAGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKA |
| | LGAPIEKTISKAKGQPREPQVYTLPPCRDELTKNQVSLWCLVKGFYPSDIAVEWESNGQPENNYKT |
| | TPPVLDSDGSFFLYSKLTVDKSRWQQGNVFSCSVMHEALHNHYTQKSLSLSPGK |
| SEQ ID NO: 3 | <Her2>pro<CD3>VH, <EGFR>pro<CD3>VH |
| | Heavy chain-scFab fusion |
| | VQLVQSGAEVKKPGASVKVSCKASGYTFTNYYIHWVREAPGQGLEWIGWIYPGDGNTKYNEKFKGR |
| | ATLTADTSTSTAYLELSSLRSEDTAVYYCARDSYSNYYFDYWGQGTLVTVSSASVAAPSVFIFPPS |
| | DEQLKSGTASVVCLLNNFYPREAKVQWKVDNALQSGNSQESVTEQDSKDSTYSLSSTLTLSKADYE |
| | KHKVYACEVTHQGLSSPVTKSFNRGESGGGGSGGGGSGGGGSGGGGSGGGGSGGSGGDIQMTQSPS |
| | SLSASVGDRVTITCRASQDVGSGVAWYQEKPGKAPKLLIGSASGLYSGVPSRFSGSRSGTDFTLTI |
| | SSLQPEDFATYYCQQSASGGSTFGQGTKVEIKSSASTKGPSVFPLAPSSKSTSGGTAALGCLVKDY |
| | FPEPVTVSWNSGALTSGVHTFPAVLQSSGLYSLSSVVTVPSSSLGTQTYICNVNHKPSNTKVDKKV |
| | EPKSSDKTHTCPPCPAPEAAGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALGAPIEKTISKAKGQPREPQ |
| | VCTLPPSRDELTKNQVSLSCAVKGFYPSDIAVEWESNGQPENNYKTTPPVLDSDGSFFLVSKLTVD |
| | KSRWQQGNVFSCSVMHEALHNHYTQKSLSLSPGK |
| SEQ ID NO: 4 | <Her2>pro<CD3>VL, <EGFR>pro<CD3>VL, <Nada>pro<CD3> VL (6G4S |
| | linker): |
| | Heavy chain-scFab fusion |
| | DIVMTQSPDSLAVSLGERATINCKSSQSLLNSRTRKNYLAWYQKKPGQPPKLLIYWASTRESGVPD |
| | RFSGSGSGTDFTLTISSLQAEDVAVYYCTQSFILRTFGQGTKVEIKSSASTKGPSVFPLAPSSKST |
| | SGGTAALGCLVKDYFPEPVTVSWNSGALTSGVHTFPAVLQSSGLYSLSSVVTVPSSSLGTQTYICN |
| | VNHKPSNTKVDKKVEPKSSGGGGSGGGGSGGGGSGGGGSGGGGSGGSGGEVQLVESGGGLVQPGGS |
| | LRLSCAASGFSIAGTAIHWVRKAPGKGLEWVASISPGGGSTAYADSVKGRFTISADTSKNTAYLQM |
| | NSLRAEDTAVYYCSRSGGSGASAMDYWGQGTLVTVSSASVAAPSVFIFPPSDEQLKSGTASVVCLL |
| | NNFYPREAKVQWKVDNALQSGNSQESVTEQDSKDSTYSLSSTLTLSKADYEKHKVYACEVTHQGLS |
| | SPVTKSFNRGESDKTHTCPPCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALGAPIEKTISKAK |
| | GQPREPQVCTLPPSRDELTKNQVSLSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID NO: 5 | <EGFR>pro<CD3>VH |
| | EGFR light chain |
| | DILLTQSPVILSVSPGERVSFSCRASQSIGTNIHWYQQRTNGSPRLLIKYASESISGIPSRFSGSG |
| | SGTDFTLSINSVESEDIADYYCQQNNNWPTTFGAGTKLELKRTVAAPSVFIFPPSDEQLKSGTASV |
| | VCLLNNFYPREAKVQWKVDNALQSGNSQESVTEQDSKDSTYSLSSTLTLSKADYEKHKVYACEVTH |
| | QGLSSPVTKSFNRGEC |
| SEQ ID NO: 6 | <EGFR>pro<CD3>VH, <EGFR>pro<CD3>VL |
| | EGFR heavy chain |
| | QVQLKQSGPGLVQPSQSLSITCTVSGFSLTNYGVHWVRQSPGKGLEWLGVIWSGGNTDYNTPFTSRL |
| | SINKDNSKSQVFFKMNSLQSNDTAIYYCARALTYYDYEFAYWGQGTLVTVSAASTKGPSVFPLAPSS |
| | KSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQDWLNGKEYKCKVSNKALGAPIE |
| | KTISKAKGQPREPQVYTLPPCRDELTKNQVSLWCLVKGFYPSDIAVEWESNGQPENNYKTTPPVLDS |
| | DGSFFLYSKLTVDKSRWQQGNVFSCSVMHEALHNHYTQKSLSLSPGK |
| SEQ ID NO: 7 | <Nada>pro<CD3> VL (6G4S linker), <Nada>pro<CD3> VL (no linker) |
| | Nada light chain |
| | DIQMTQSPSSLSASVGDRVTITCRASQDVGSGVAWYQQKPGKAPKLLIGSASGLYSGVPSRFSGSRS |
| | GTDFTLTISSLQPEDFATYYCQQSASGGSTFGQGTKVEIKRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC |
| SEQ ID NO: 8 | <Nada>pro<CD3> VL (6G4S linker), <Nada>pro<CD3> VL (no linker) |
| | Nada heavy chain |
| | EVQLVESGGGLVQPGGSLRLSCAASGFSIAGTAIHWVRQAPGKGLEWVASISPGGGSTAYADSVKGR |
| | FTISADTSKNTAYLQMNSLRAEDTAVYYCSRSGGSGASAMDYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQTY |
| | ICNVNHKPSNTKVDKKVEPKSCDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALGAPI |
| | EKTISKAKGQPREPQVYTLPPCRDELTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK |
| SEQ ID NO: 9 | <Her2>pro<CD3> VH (no linker) |
| | Heavy chain CD3 VH |
| | EVQLVQSGAEVKKPGASVKVSCKASGYTFTNYYIHWVREAPGQGLEWIGWIYPGDGNTKYNEKFKGR |
| | ATLTADTSTSTAYLELSSLRSEDTAVYYCARDSYSNYYFDYWGQGTLVTVSSASVAAPSVFIFPPSD |
| | EQLKSGTASVVCLLNNFYPREAKVQWKVDNALQSGNSQESVTEQDSKDSTYSLSSTLTLSKADYEKH |
| | KVYACEVTHQGLSSPVTKSFNRGESDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWINGKEYKCKVSNKALG |
| | APIEKTISKAKGQPREPQVCTLPPSRDELTKNQVSLSCAVKGFYPSDIAVEWESNGQPENNYKTTPP |
| | VLDSDGSFFLVSKLTVDKSRWQQGNVFSCSVMHEALHNHYTQKSLSLSPGK |
| SEQ ID NO: 10 | <Her2>pro<CD3> VH (no linker) |
| | Light chain Nada VL |
| | DIQMTQSPSSLSASVGDRVTITCRASQDVGSGVAWYQEKPGKAPKLLIGSASGLYSGVPSRFSGSRS |
| | GTDFTLTISSLQPEDFATYYCQQSASGGSTFGQGTKVEIKSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNT |
| | KVDKKVEPKSS |
| SEQ ID NO: 11 | <Her2>pro<CD3> VL (no linker), <Nada>pro<CD3> VL (no linker) |
| | Heavy chain CD3 VL |
| | DIVMTQSPDSLAVSLGERATINCKSSQSLLNSRTRKNYLAWYQKKPGQPPKLLIYWASTRESGVPDR |
| | FSGSGSGTDFTLTISSLQAEDVAVYYCTQSFILRTFGQGTKVEIKSSASTKGPSVFPLAPSSKSTSG |
| | GTAALGCLVKDYFPEPVTVSWNSGALTSGVHTFPAVLQSSGLYSLSSVVTVPSSSLGTQTYICNVNH |
| | KPSNTKVDKKVEPKSSDKTHTCPPCPAPEAAGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALGAPIEKTISK |
| | AKGQPREPQVCTLPPSRDELTKNQVSLSCAVKGFYPSDIAVEWESNGQPENNYKTTPPVLDSDGSFF |
| | LVSKLTVDKSRWQQGNVFSCSVMHEALHNHYTQKSLSLSPGK |
| SEQ ID NO: 12 | <Her2>pro<CD3> VL (no linker), <Nada>pro<CD3> VL (no linker) |
| | Light chain Nada VH |
| | EVQLVESGGGLVQPGGSLRLSCAASGFSIAGTAIHWVRKAPGKGLEWVASISPGGGSTAYADSVKGR |
| | FTISADTSKNTAYLQMNSLRAEDTAVYYCSRSGGSGASAMDYWGQGTLVTVSSASVAAPSVFIFPPS |
| | DEQLKSGTASVVCLLNNFYPREAKVQWKVDNALQSGNSQESVTEQDSKDSTYSLSSTLTLSKADYEK |
| | HKVYACEVTHQGLSSPVTKSFNRGES |
| SEQ ID NO: 13 | <Her2>(Dig)(CD3) [A] |
| | Heavy chain-scFab fusion |
| | EVQLVQSGAEVKKPGASVKVSCKASGYTFTNYYIHWVREAPGQGLEWIGWIYPGDGNTKYNEKFKGR |
| | ATLTADTSTSTAYLELSSLRSEDTAVYYCARDSYSNYYFDYWGQGTLVTVSSASVAAPSVFIFPPSD |
| | EQLKSGTASVVCLLNNFYPREAKVQWKVDNALQSGNSQESVTEQDSKDSTYSLSSTLTLSKADYEKH |
| | KVYACEVTHQGLSSPVTKSFNRGESGGGGSGGGGSGGGGSGGGGSGGGGSGGSGGDIQMTQSPSSLS |
| | ASVGDRVTITCRASQDIKNYLNWYQEKPGKAPKLLIYYSSTLLSGVPSRFSGSGSGTDFTLTISSLQ |
| | PEDFATYYCQQSITLPPTFGGGTKVEIKSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPSNTKVDKKVEPKSSD |
| | KTHTCPPCPAPEAAGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALGAPIEKTISKAKGQPREPQVCTLPPSR |
| | DELTKNQVSLSCAVKGFYPSDIAVEWESNGQPENNYKTTPPVLDSDGSFFLVSKLTVDKSRWQQGNV |
| | FSCSVMHEALHNRFTQKSLSLSPGK |
| SEQ ID NO: 14 | <Her2>(Dig)(CD3) [B] |
| | Heavy chain-scFab fusion |
| | DIVMTQSPDSLAVSLGERATINCKSSQSLLNSRTRKNYLAWYQKKPGQPPKLLIYWASTRESGVPDR |
| | FSGSGSGTDFTLTISSLQAEDVAVYYCTQSFILRTFGQGTKVEIKSSASTKGPSVFPLAPSSKSTSG |
| | GTAALGCLVKDYFPEPVTVSWNSGALTSGVHTFPAVLQSSGLYSLSSVVTVPSSSLGTQTYICNVNH |
| | KPSNTKVDKKVEPKSSGGGGSGGGGSGGGGSGGGGSGGGGSGGSGGQVQLVESGGGLVKPGGSLRLS |
| | CAASGFTFSDYAMSWIRKAPGKGLEWVSSINIGATYIYYADSVKGRFTISRDNAKNSLYLQMNSLRA |
| | EDTAVYYCARPGSPYEYDKAYYSMAYWGQGTTVTVSSASVAAPSVFIFPPSDEQLKSGTASVVCLLN |
| | NFYPREAKVQWKVDNALQSGNSQESVTEQDSKDSTYSLSSTLTLSKADYEKHKVYACEVTHQGLSSP |
| | VTKSFNRGESDKTHTCPPCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALGAPIEKTISKAKGQPR |
| | EPQVCTLPPSRDELTKNQVSLSCAVKGFYPSDIAVEWESNGQPENNYKTTPPVLDSDGSFFLVSKLT |
| | VDKSRWQQGNVFSCSVMHEALHNRFTQKSLSLSPGK |
| SEQ ID NO: 15 | <Her2>(CD3)(Dig) [A] |
| | Heavy chain-scFab fusion |
| | QVQLVESGGGLVKPGGSLRLSCAASGFTFSDYAMSWIREAPGKGLEWVSSINIGATYIYYADSVKGR |
| | FTISRDNAKNSLYLQMNSLRAEDTAVYYCARPGSPYEYDKAYYSMAYWGQGTTVTVSSASVAAPSVF |
| | IFPPSDEQLKSGTASVVCLLNNFYPREAKVQWKVDNALQSGNSQESVTEQDSKDSTYSLSSTLTLSK |
| | ADYEKHKVYACEVTHQGLSSPVTKSFNRGESGGGGSGGGGSGGGGSGGGGSGGGGSGGSGGDIVMTQ |
| | SPDSLAVSLGERATINCKSSQSLLNSRTRKNYLAWYQEKPGQPPKLLIYWASTRESGVPDRFSGSGS |
| | GTDFTLTISSLQAEDVAVYYCTQSFILRTFGQGTKVEIKSSASTKGPSVFPLAPSSKSTSGGTAALG |
| | CLVKDYFPEPVTVSWNSGALTSGVHTFPAVLQSSGLYSLSSVVTVPSSSLGTQTYICNVNHKPSNTK |
| | VDKKVEPKSSDKTHTCPPCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALGAPIEKTISKAKGQPR |
| | EPQVCTLPPSRDELTKNQVSLSCAVKGFYPSDIAVEWESNGQPENNYKTTPPVLDSDGSFFLVSKLT |
| | VDKSRWQQGNVFSCSVMHEALHNRFTQKSLSLSPGK |
| SEQ ID NO: 16 | <Her2>(CD3)(Dig) [B] |
| | Heavy chain-scFab fusion |
| | DIQMTQSPSSLSASVGDRVTITCRASQDIKNYLNWYQKKPGKAPKLLIYYSSTLLSGVPSRFSGSGS |
| | GTDFTLTISSLQPEDFATYYCQQSITLPPTFGGGTKVEIKSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNT |
| | KVDKKVEPKSSGGGGSGGGGSGGGGSGGGGSGGGGSGGSGGEVQLVQSGAEVKKPGASVKVSCKASG |
| | YTFTNYYIHWVRKAPGQGLEWIGWIYPGDGNTKYNEKFKGRATLTADTSTSTAYLELSSLRSEDTAV |
| | YYCARDSYSNYYFDYWGQGTLVTVSSASVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGESD |
| | KTHTCPPCPAPEAAGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALGAPIEKTISKAKGQPREPQVCTLPPSR |
| | DELTKNQVSLSCAVKGFYPSDIAVEWESNGQPENNYKTTPPVLDSDGSFFLVSKLTVDKSRWQQGNV |
| | FSCSVMHEALHNRFTQKSLSLSPGK |
| SEQ ID NO: 17 | <Her2>(CD3)(Nada) [A] |
| | Heavy chain-scFab fusion |
| | EVQLVESGGGLVQPGGSLRLSCAASGFSIAGTAIHWVREAPGKGLEWVASISPGGGSTAYADSVKGR |
| | FTISADTSKNTAYLQMNSLRAEDTAVYYCSRSGGSGASAMDYWGQGTLVTVSSASVAAPSVFIFPPS |
| | DEQLKSGTASVVCLLNNFYPREAKVQWKVDNALQSGNSQESVTEQDSKDSTYSLSSTLTLSKADYEK |
| | HKVYACEVTHQGLSSPVTKSFNRGESGGGGSGGGGSGGGGSGGGGSGGGGSGGSGGDIVMTQSPDSL |
| | AVSLGERATINCKSSQSLLNSRTRKNYLAWYQEKPGQPPKLLIYWASTRESGVPDRFSGSGSGTDFT |
| | LTISSLQAEDVAVYYCTQSFILRTFGQGTKVEIKSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKV |
| | EPKSSDKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALGAPIEKTISKAKGQPREPQVC |
| | TLPPSRDELTKNQVSLSCAVKGFYPSDIAVEWESNGQPENNYKTTPPVLDSDGSFFLVSKLTVDKSR |
| | WQQGNVFSCSVMHEALHNRFTQKSLSLSPGK |
| SEQ ID NO: 18 | <Her2>(CD3)(Nada) [B] |
| | Heavy chain-scFab fusion |
| | DIQMTQSPSSLSASVGDRVTITCRASQDVGSGVAWYQKKPGKAPKLLIGSASGLYSGVPSRFSGSRS |
| | GTDFTLTISSLQPEDFATYYCQQSASGGSTFGQGTKVEIKSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNT |
| | KVDKKVEPKSSGGGGSGGGGSGGGGSGGGGSGGGGSGGSGGEVQLVQSGAEVKKPGASVKVSCKASG |
| | YTFTNYYIHWVRKAPGQGLEWIGWIYPGDGNTKYNEKFKGRATLTADTSTSTAYLELSSLRSEDTAV |
| | YYCARDSYSNYYFDYWGQGTLVTVSSASVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGESD |
| | KTHTCPPCPAPEAAGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALGAPIEKTISKAKGQPREPQVCTLPPSR |
| | DELTKNQVSLSCAVKGFYPSDIAVEWESNGQPENNYKTTPPVLDSDGSFFLVSKLTVDKSRWQQGNV |
| | FSCSVMHEALHNRFTQKSLSLSPGK |
| SEQ ID NO: 19 | <HER2>(CD28)(CD3) [A] |
| | EVQLVQSGAEVKKPGASVKVSCKASGYTFTNYYIHWVREAPGQGLEWIGWIYPGDGNTKYNEKFKGR |
| | ATLTADTSTSTAYLELSSLRSEDTAVYYCARDSYSNYYFDYWGQGTLVTVSSASVAAPSVFIFPPSD |
| | EQLKSGTASVVCLLNNFYPREAKVQWKVDNALQSGNSQESVTEQDSKDSTYSLSSTLTLSKADYEKH |
| | KVYACEVTHQGLSSPVTKSFNRGESGGGGSGGGGSGGGGSGGGGSGGGGSGGGGSGGSGGDIQMTQS |
| | PSSLSASVGDRVTITCHASQNIYVWLNWYQEKPGKAPKLLIYKASNLHTGVPSRFSGSGSGTDFTLT |
| | ISSLQPEDFATYYCQQGQTYPYTFGGGTKVEIKSSASTKGPSVFPLAPSSKSTSGGTAALGCLVKDY |
| | FPEPVTVSWNSGALTSGVHTFPAVLQSSGLYSLSSVVTVPSSSLGTQTYICNVNHKPSNTKVDKKVE |
| | PKSSDKTHTCPPCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALGAPIEKTISKAKGQPREPQVCT |
| | LPPSRDELTKNQVSLSCAVKGFYPSDIAVEWESNGQPENNYKTTPPVLDSDGSFFLVSKLTVDKSRW |
| | QQGNVFSCSVMHEALHNHYTQKSLSLSPGK |
| SEQ ID NO: 20 | <HER2>(CD28)(CD3) [B] |
| | DIVMTQSPDSLAVSLGERATINCKSSQSLLNSRTRKNYLAWYQKKPGQPPKLLIYWASTRESGVPDR |
| | FSGSGSGTDFTLTISSLQAEDVAVYYCTQSFILRTFGQGTKVEIKSSASTKGPSVFPLAPSSKSTSG |
| | GTAALGCLVKDYFPEPVTVSWNSGALTSGVHTFPAVLQSSGLYSLSSVVTVPSSSLGTQTYICNVNH |
| | KPSNTKVDKKVEPKSSGGGGSGGGGSGGGGSGGGGSGGGGSGGGGSGGSGGQVQLVQSGAEVKKPGA |
| | SVKVSCKASGYTFTSYYIHWVRKAPGQGLEWIGCIYPGNVNTNYNEKFKDRATLTVDTSISTAYMEL |
| | SRLRSDDTAVYFCTRSHYGLDWNFDVWGQGTTVTVSSASVAAPSVFIFPPSDEQLKSGTASVVCLLN |
| | NFYPREAKVQWKVDNALQSGNSQESVTEQDSKDSTYSLSSTLTLSKADYEKHKVYACEVTHQGLSSP |
| | VTKSFNRGESDKTHTCPPCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALGAPIEKTISKAKGQPR |
| | EPQVCTLPPSRDELTKNQVSLSCAVKGFYPSDIAVEWESNGQPENNYKTTPPVLDSDGSFFLVSKLT |
| | VDKSRWQQGNVFSCSVMHEALHNHYTQKSLSLSPGK |
| SEQ ID NO: 21 | <HER2>(CD3)(NADA) [A] |
| | EVQLVESGGGLVQPGGSLRLSCAASGFSIAGTAIHWVREAPGKGLEWVASISPGGGSTAYADSVKGR |
| | FTISADTSKNTAYLQMNSLRAEDTAVYYCSRSGGSGASAMDYWGQGTLVTVSSASVAAPSVFIFPPS |
| | DEQLKSGTASVVCLLNNFYPREAKVQWKVDNALQSGNSQESVTEQDSKDSTYSLSSTLTLSKADYEK |
| | HKVYACEVTHQGLSSPVTKSFNRGESGGGGSGGGGSGGGGSGGGGSGGGGSGGSGGDIVMTQSPDSL |
| | AVSLGERATINCKSSQSLLNSRTRKNYLAWYQEKPGQPPKLLIYWASTRESGVPDRFSGSGSGTDFT |
| | LTISSLQAEDVAVYYCTQSFILRTFGQGTKVEIKSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKV |
| | EPKSSDKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALGAPIEKTISKAKGQPREPQVC |
| | TLPPSRDELTKNQVSLSCAVKGFYPSDIAVEWESNGQPENNYKTTPPVLDSDGSFFLVSKLTVDKSR |
| | WQQGNVFSCSVMHEALHNRFTQKSLSLSPGK |
| SEQ ID NO: 22 | <HER2>(CD3)(NADA) [B] |
| | DIQMTQSPSSLSASVGDRVTITCRASQDVGSGVAWYQKKPGKAPKLLIGSASGLYSGVPSRFSGSRS |
| | GTDFTLTISSLQPEDFATYYCQQSASGGSTFGQGTKVEIKSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNT |
| | KVDKKVEPKSSGGGGSGGGGSGGGGSGGGGSGGGGSGGSGGEVQLVQSGAEVKKPGASVKVSCKASG |
| | YTFTNYYIHWVRKAPGQGLEWIGWIYPGDGNTKYNEKFKGRATLTADTSTSTAYLELSSLRSEDTAV |
| | YYCARDSYSNYYFDYWGQGTLVTVSSASVAAPSVFIFPPSDEQLKSGTASVVCLLNNFYPREAKVQW |
| | KVDNALQSGNSQESVTEQDSKDSTYSLSSTLTLSKADYEKHKVYACEVTHQGLSSPVTKSFNRGESD |
| | KTHTCPPCPAPEAAGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALGAPIEKTISKAKGQPREPQVCTLPPSR |
| | DELTKNQVSLSCAVKGFYPSDIAVEWESNGQPENNYKTTPPVLDSDGSFFLVSKLTVDKSRWQQGNV |
| | FSCSVMHEALHNRFTQKSLSLSPGK |
| SEQ ID NO: 23 | <HER2>(CD3)(CD28) [A] |
| | QVQLVQSGAEVKKPGASVKVSCKASGYTFTSYYIHWVREAPGQGLEWIGCIYPGNVNTNYNEKFKDR |
| | ATLTVDTSISTAYMELSRLRSDDTAVYFCTRSHYGLDWNFDVWGQGTTVTVSSASVAAPSVFIFPPS |
| | DEQLKSGTASVVCLLNNFYPREAKVQWKVDNALQSGNSQESVTEQDSKDSTYSLSSTLTLSKADYEK |
| | HKVYACEVTHQGLSSPVTKSFNRGESGGGGSGGGGSGGGGSGGGGSGGGGSGGGGSGGSGGDIVMTQ |
| | SPDSLAVSLGERATINCKSSQSLLNSRTRKNYLAWYQEKPGQPPKLLIYWASTRESGVPDRFSGSGS |
| | GTDFTLTISSLQAEDVAVYYCTQSFILRTFGQGTKVEIKSSASTKGPSVFPLAPSSKSTSGGTAALG |
| | CLVKDYFPEPVTVSWNSGALTSGVHTFPAVLQSSGLYSLSSVVTVPSSSLGTQTYICNVNHKPSNTK |
| | VDKKVEPKSSDKTHTCPPCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALGAPIEKTISKAKGQPR |
| | EPQVCTLPPSRDELTKNQVSLSCAVKGFYPSDIAVEWESNGQPENNYKTTPPVLDSDGSFFLVSKLT |
| | VDKSRWQQGNVFSCSVMHEALHNHYTQKSLSLSPGK |
| SEQ ID NO: 24 | <HER2>(CD3)(CD28) [B] |
| | DIQMTQSPSSLSASVGDRVTITCHASQNIYVWLNWYQKKPGKAPKLLIYKASNLHTGVPSRFSGSGS |
| | GTDFTLTISSLQPEDFATYYCQQGQTYPYTFGGGTKVEIKSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNT |
| | KVDKKVEPKSSGGGGSGGGGSGGGGSGGGGSGGGGSGGGGSGGSGGEVQLVQSGAEVKKPGASVKVS |
| | CKASGYTFTNYYIHWVRKAPGQGLEWIGWIYPGDGNTKYNEKFKGRATLTADTSTSTAYLELSSLRS |
| | EDTAVYYCARDSYSNYYFDYWGQGTLVTVSSASVAAPSVFIFPPSDEQLKSGTASVVCLLNNFYPRE |
| | AKVQWKVDNALQSGNSQESVTEQDSKDSTYSLSSTLTLSKADYEKHKVYACEVTHQGLSSPVTKSFN |
| | RGESDKTHTCPPCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALGAPIEKTISKAKGQPREPQVCT |
| | LPPSRDELTKNQVSLSCAVKGFYPSDIAVEWESNGQPENNYKTTPPVLDSDGSFFLVSKLTVDKSRW |
| | QQGNVFSCSVMHEALHNHYTQKSLSLSPGK |
| SEQ ID NO: 25 | PD-1 0376 LC: |
| | DIVMTQSPDSLAVSLGERATINCKASESVDTSDNSFIHWYQQKPGQSPKLLIYRSSTLESGVPDRFS |
| | GSGSGTDFTLTISSLQAEDVAVYYCQQNYDVPWTFGQGTKVEIKRTVAAPSVFIFPPSDEQLKSGTA |
| | SVVCLLNNFYPREAKVQWKVDNALQSGNSQESVTEQDSKDSTYSLSSTLTLSKADYEKHKVYACEVT |
| | HQGLSSPVTKSFNRGEC |
| SEQ ID NO: 26 | PD-1 0376 HC: |
| | EVQLLESGGGLVQPGGSLRLSCAASGFSFSSYTMSWVRQAPGKGLEWVATISGGGRDIYYPDSVKGR |
| | FTISRDNSKNTLYLQMNSLRAEDTAVYYCVLLTGRVYFALDSWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQTY |
| | ICNVNHKPSNTKVDKKVEPKSCDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALGAPI |
| | EKTISKAKGQPREPQVYTLPPCRDELTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK |
| SEQ ID NO: 27 | XVV142 scFab HC (IL2Rβ Mik-β1VH outer position, IL2Rγ Cl.3 VL inner |
| | position) |
| | QVQLKQSGPGLVQPSQSLSITCTVSGFSVTSYGVHWIRESPGKGLEWLGVIWSGGSTDYNAAFISRL |
| | TISKDNSKSQVFFKVNSLQPADTAIYYCARAGDYNYDGFAYWGQGTLVTVSSASVAAPSVFIFPPSD |
| | EQLKSGTASVVCLLNNFYPREAKVQWKVDNALQSGNSQESVTEQDSKDSTYSLSSTLTLSKADYEKH |
| | KVYACEVTHQGLSSPVTKSFNRGESGGGGSGGGGSGGGGSGGGGSGGGGSGGSGGDIQMTQSPSTLS |
| | ASVGDRVTITCRASQSISSWLAWYQEKPGKAPKLLIYDASSLESGVPSRFSGSGSGTEFTLTISSLQ |
| | PDDFATYYCQQYSSYYTFGQGTKVEIKSSASTKGPSVFPLAPSSKSTSGGTAALGCLVKDYFPEPVT |
| | VSWNSGALTSGVHTFPAVLQSSGLYSLSSVVTVPSSSLGTQTYICNVNHKPSNTKVDKKVEPKSSDK |
| | THTCPPCPAPEAAGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALGAPIEKTISKAKGQPREPQVCTLPPSRD |
| | ELTKNQVSLSCAVKGFYPSDIAVEWESNGQPENNYKTTPPVLDSDGSFFLVSKLTVDKSRWQQGNVF |
| | SCSVMHEALHNRFTQKSLSLSPGK |
| SEQ ID NO: 28 | XVV143 scFab HC (IL2Rβ Cl.4 VH outer position, IL2Rγ Cl.1 VL inner |
| | position) |
| | QVQLVQSGAEVKKPGASVKVSCKASGYTFTSYYMHWVREAPGQGLEWMGIINPSGGSTSYAQKFQGR |
| | VTMTRDTSTSTVYMELSSLRSEDTAVYYCARDYSWWGDSYTGFDYWGQGTLVTVSSASVAAPSVFIF |
| | PPSDEQLKSGTASVVCLLNNFYPREAKVQWKVDNALQSGNSQESVTEQDSKDSTYSLSSTLTLSKAD |
| | YEKHKVYACEVTHQGLSSPVTKSFNRGESGGGGSGGGGSGGGGSGGGGSGGGGSGGSGGDIQMTQSP |
| | STLSASVGDRVTITCRASQSISSWLAWYQEKPGKAPKLLIYDASSLESGVPSRFSGSGSGTEFTLTI |
| | SSLQPDDFATYYCQQYNSDWWTFGQGTKVEIKSSASTKGPSVFPLAPSSKSTSGGTAALGCLVKDYF |
| | PEPVTVSWNSGALTSGVHTFPAVLQSSGLYSLSSVVTVPSSSLGTQTYICNVNHKPSNTKVDKKVEP |
| | KSSDKTHTCPPCPAPEAAGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALGAPIEKTISKAKGQPREPQVCTL |
| | PPSRDELTKNQVSLSCAVKGFYPSDIAVEWESNGQPENNYKTTPPVLDSDGSFFLVSKLTVDKSRWQ |
| | QGNVFSCSVMHEALHNRFTQKSLSLSPGK |
| SEQ ID NO: 29 | XVV144 scFab HC (IL2Rβ Cl.4 VH outer position, IL2Rγ Cl.3 VL inner |
| | position) |
| | QVQLVQSGAEVKKPGASVKVSCKASGYTFTSYYMHWVREAPGQGLEWMGIINPSGGSTSYAQKFQGR |
| | VTMTRDTSTSTVYMELSSLRSEDTAVYYCARDYSWWGDSYTGFDYWGQGTLVTVSSASVAAPSVFIF |
| | PPSDEQLKSGTASVVCLLNNFYPREAKVQWKVDNALQSGNSQESVTEQDSKDSTYSLSSTLTLSKAD |
| | YEKHKVYACEVTHQGLSSPVTKSFNRGESGGGGSGGGGSGGGGSGGGGSGGGGSGGSGGDIQMTQSP |
| | STLSASVGDRVTITCRASQSISSWLAWYQEKPGKAPKLLIYDASSLESGVPSRFSGSGSGTEFTLTI |
| | SSLQPDDFATYYCQQYSSYYTFGQGTKVEIKSSASTKGPSVFPLAPSSKSTSGGTAALGCLVKDYFP |
| | EPVTVSWNSGALTSGVHTFPAVLQSSGLYSLSSVVTVPSSSLGTQTYICNVNHKPSNTKVDKKVEPK |
| | SSDKTHTCPPCPAPEAAGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALGAPIEKTISKAKGQPREPQVCTLP |
| | PSRDELTKNQVSLSCAVKGFYPSDIAVEWESNGQPENNYKTTPPVLDSDGSFFLVSKLTVDKSRWQQ |
| | GNVFSCSVMHEALHNRFTQKSLSLSPGK |
| SEQ ID NO: 30 | PD-1 1040 LC: |
| | DIQMTQSPSSLSASVGDRVTITCRASQSISSYLNWYQQKPGKAPKLLIYTASSLQSGVPSRFSGSGS |
| | GTDFTLTISSLQPEDFATYYCQQSYSTPLTFGGGTKVEIKRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC |
| SEQ ID NO: 31 | PD-1 1040 HC: |
| | EVQLLESGGGLVQPGGSLRLSCAASGFTFSSYAMSWVRQAPGKGLEWVSVITGSGGSTYYADSVKGR |
| | FTISRDNSRNTLYLQMNSLRAEDTAVYYCAKGEGYAGSSYFRASDIWGQGTMVTVSSASTKGPSVFP |
| | LAPSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPEAAGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKAL |
| | GAPIEKTISKAKGQPREPQVYTLPPCRDELTKNQVSLWCLVKGFYPSDIAVEWESNGQPENNYKTTP |
| | PVLDSDGSFFLYSKLTVDKSRWQQGNVFSCSVMHEALHNHYTQKSLSLSPGK |
| SEQ ID NO: 32 | XVV146 scFab HC (IL2Rβ Mik-β1 VL outer position, IL2Rγ Cl.3 VH |
| | inner position) |
| | QIVLTQSPAIMSASPGEKVTMTCSGSSSVSFMYWYQKRPGSSPRLLIYDTSNLASGVPVRFSGSGSG |
| | TSYSLTISRMEAEDAATYYCQQWSTYPLTFGAGTKLELKSSASTKGPSVFPLAPSSKSTSGGTAALG |
| | CLVKDYFPEPVTVSWNSGALTSGVHTFPAVLQSSGLYSLSSVVTVPSSSLGTQTYICNVNHKPSNTK |
| | VDKKVEPKSSGGGGSGGGGSGGGGSGGGGSGGGGSGGSGGEVQLLESGGGLVQPGGSLRLSCAASGF |
| | TFSSYAMSWVRKAPGKGLEWVSAISGSGGSTYYADSVKGRFTISRDNSKNTLYLQMNSLRAEDTAVY |
| | YCARLLYYGFDYWGQGTLVTVSSASVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLSSPVTKSFNRGESDKTH |
| | TCPPCPAPEAAGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALGAPIEKTISKAKGQPREPQVCTLPPSRDEL |
| | TKNQVSLSCAVKGFYPSDIAVEWESNGQPENNYKTTPPVLDSDGSFFLVSKLTVDKSRWQQGNVFSC |
| | SVMHEALHNRFTQKSLSLSPGK |
| SEQ ID NO: 33 | XVV147 scFab HC (IL2Rβ Cl.4 VL outer position, IL2Rγ Cl.1 VH inner |
| | position) |
| | DIVMTQSPLSLPVTPGEPASISCRSSQSLLHSNGYNYLDWYLKKPGQSPQLLIYLGSNRASGVPDRF |
| | SGSGSGTDFTLKISRVEAEDVGVYYCMQVLQPPPTFGQGTKVEIKSSASTKGPSVFPLAPSSKSTSG |
| | GTAALGCLVKDYFPEPVTVSWNSGALTSGVHTFPAVLQSSGLYSLSSVVTVPSSSLGTQTYICNVNH |
| | KPSNTKVDKKVEPKSSGGGGSGGGGSGGGGSGGGGSGGGGSGGSGGQVQLVQSGAEVKKPGSSVKVS |
| | CKASGGTFSSYAISWVRKAPGQGLEWMGGIIPIFGTANYAQKFQGRVTITADKSTSTAYMELSSLRS |
| | EDTAVYYCARDPGPPYNWYWGAYDYWGQGTTVTVSSASVAAPSVFIFPPSDEQLKSGTASVVCLLNN |
| | FYPREAKVQWKVDNALQSGNSQESVTEQDSKDSTYSLSSTLTLSKADYEKHKVYACEVTHQGLSSPV |
| | TKSFNRGESDKTHTCPPCPAPEAAGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALGAPIEKTISKAKGQPRE |
| | PQVCTLPPSRDELTKNQVSLSCAVKGFYPSDIAVEWESNGQPENNYKTTPPVLDSDGSFFLVSKLTV |
| | DKSRWQQGNVFSCSVMHEALHNRFTQKSLSLSPGK |
| SEQ ID NO: 34 | XVV148 scFab HC (IL2Rβ Cl.4 VL outer position, IL2Rγ Cl.3 VH inner |
| | position) |
| | DIVMTQSPLSLPVTPGEPASISCRSSQSLLHSNGYNYLDWYLKKPGQSPQLLIYLGSNRASGVPDRF |
| | SGSGSGTDFTLKISRVEAEDVGVYYCMQVLQPPPTFGQGTKVEIKSSASTKGPSVFPLAPSSKSTSG |
| | GTAALGCLVKDYFPEPVTVSWNSGALTSGVHTFPAVLQSSGLYSLSSVVTVPSSSLGTQTYICNVNH |
| | KPSNTKVDKKVEPKSSGGGGSGGGGSGGGGSGGGGSGGGGSGGSGGEVQLLESGGGLVQPGGSLRLS |
| | CAASGFTFSSYAMSWVRKAPGKGLEWVSAISGSGGSTYYADSVKGRFTISRDNSKNTLYLQMNSLRA |
| | EDTAVYYCARLLYYGFDYWGQGTLVTVSSASVAAPSVFIFPPSDEQLKSGTASVVCLLNNFYPREAK |
| | VQWKVDNALQSGNSQESVTEQDSKDSTYSLSSTLTLSKADYEKHKVYACEVTHQGLSSPVTKSFNRG |
| | ESDKTHTCPPCPAPEAAGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALGAPIEKTISKAKGQPREPQVCTLP |
| | PSRDELTKNQVSLSCAVKGFYPSDIAVEWESNGQPENNYKTTPPVLDSDGSFFLVSKLTVDKSRWQQ |
| | GNVFSCSVMHEALHNRFTQKSLSLSPGK |

### EXAMPLES

The following examples are provided to aid the understanding of the present invention, the true scope of which is set forth in the appended claims. It is understood that modifications can be made in the procedures set forth without departing from the spirit of the invention.

### Example 1: Design of (Inactive) Fusion Polypeptides Comprising Single Chain Fab Fragments and Mode of Action

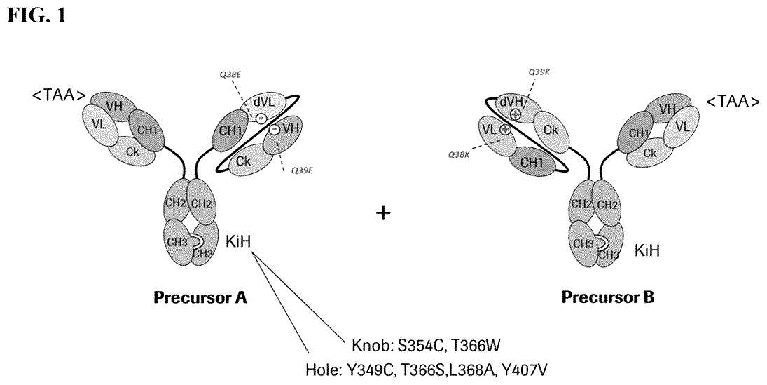

FIG. 1 shows the general design of a pair of recombinant binding proteins of the invention. In this example, IgG-shaped molecules are provided that are composed of three individual polypeptide chains: one light chain (e.g. a full length light chain comprising a light chain variable domain and a light chain constant domain), one heavy chain (e.g. a full length heavy chain comprising a heavy chain variable domain and all heavy chain constant domains including a hinge region) and one heavy chain polypeptide comprising a part of an Fc Doman (e.g. a heavy chain Fc-region fragment comprising hinge-CH2-CH3) that is fused with its N-terminus to a scFab-polypeptide. The variable domains of the light chain and the heavy chain form an antigen binding domain, here an antigen binding site. The heavy chain (e.g. derived from the human IgG1 subclass) contains knob-into-hole mutations (e.g., the mutations T366W and S354C in the CH3 domain of an antibody heavy chain is denoted as “knob” and the mutations T366S, L368A, Y407V, Y349C in the CH3 domain of an antibody heavy chain are denoted as “hole”. In the first fusion polypeptide (“precursor A”), the scFab-polypeptide comprises a domain crossover of the VH and the VL domain to ensure correct light chain pairing (N--VH-Ck-linker-dVL-CH1--C). The complementing domain comprises a variable light chain domain dVL, the linker consists of 6×(G4S) units and the first part of the effector domain is a VH domain, e.g. originating from a CD3 binding entity. Repulsive charges between dVL and VH were introduced (dVL: Q38E; VH: Q39E) to yield a partially flawed interface between the two domains. In the second fusion protein (“precursor B”), the scFab-polypeptide comprises a domain crossover of the CH1 and CL domain to ensure correct light chain pairing (N--VL-CH1-linker-dVH-Ck--C). The complementing domain comprises a variable heavy chain domain dVH, the linker consists of 6×(G4S) units and the second part of the effector domain is a VL, e.g. originating from a CD3 binding entity. Repulsive charges between dVH and VL were introduced (VL: Q38K; dVH: Q39K) to yield a partially destabilized interface between the two domains.

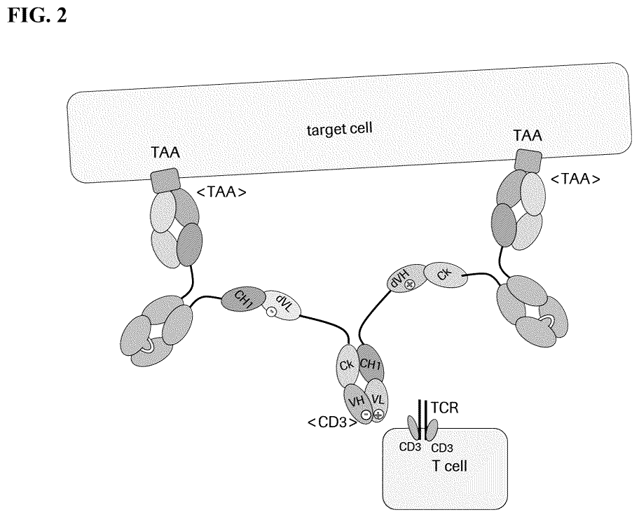

FIG. 2 shows the principle of activating the effector domain upon binding of the first and the second fusion polypeptide on target cells via their first and second antigen binding domains that bind to the same epitope on the surface of the target cell. The partially destabilized interfaces in the first and second fusion polypeptide triggers the dissociation of the <CD3>-derived VH/VL chains. Driven by the attracting charges (VH, Q39E (Precursor A); VL, Q38K (Precursor B)) an active CD3-binding Fab is generated from precursors in close proximity and mediates the engagement and activation of T cells.

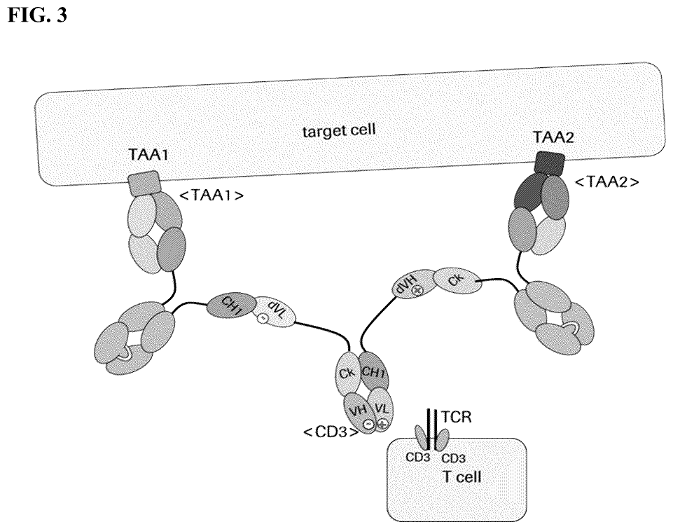

FIG. 3 shows the principle of activating the effector domain upon binding of the first and the second fusion polypeptide on target cells via their first and second antigen binding domains that bind to the different epitopes on the surface of the target cell. The partially destabilized interfaces in the prodrug modules triggers the dissociation of the <CD3>-derived VH/VL chains. Driven by the attracting charges (VH,Q39E (Precursor A); VL, Q38K (Precursor B)) an active CD3-binding Fab is generated from precursors in close proximity and mediates the engagement and activation of T cells.

### Example 2: Expression of (Inactive) Fusion Polypeptides Comprising Single Chain Fab Fragments

For expression of the fusion polypeptides used in the following examples, the following method was employed:

Expression of the first and the second fusion polypeptide was achieved by co-transfection of plasmids encoding light chain, heavy chain (with knob or hole-mutations) and matching heavy chain polypeptides comprising the part of the effector domain and the complementing domain (hole or knob) into mammalian cells (e.g. HEK293) via state of the art technologies.

In more detail, for example, for the production of the first and the second fusion polypeptide by transient transfection (e.g. in HEK293 cells) expression plasmids based either on a cDNA organization with or without a CMV-Intron A promoter or on a genomic organization with a CMV promoter were applied.

Beside the antibody expression cassettes, the plasmids contained:
an origin of replication, which allows replication of this plasmid in E. coli,
a ß-lactamase gene, which confers ampicillin resistance in E. coli, and
the dihydrofolate reductase gene from Mus musculus as a selectable marker in eukaryotic cells.

The transcription unit of each antibody gene was composed of the following elements:
unique restriction site(s) at the 5′-end
the immediate early enhancer and promoter from the human cytomegalovirus,
followed by the Intron A sequence in the case of the cDNA organization,
a 5′-untranslated region of a human antibody gene,
an immunoglobulin heavy chain signal sequence,
the antibody chain either as cDNA or in genomic organization (the immunoglobulin exon-intron organization is maintained),
a 3′-non-translated region with a polyadenylation signal sequence, and
unique restriction site(s) at the 3′-end.

The fusion genes comprising the antibody chains were generated by gene synthesis and assembled by known recombinant methods and techniques by connection of the according nucleic acid segments e.g. using unique restriction sites in the respective plasmids. The subcloned nucleic acid sequences were verified by DNA sequencing. For transient transfections larger quantities of the plasmids were prepared by plasmid preparation from transformed E. coli cultures (Nucleobond AX, Macherey-Nagel).

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

The first and the second fusion polypeptide were generated by transient transfection with the respective plasmid using the HEK293-Expi system (ThermoFisher) according to the manufacturer's instruction. Because the fusion polypeptides contain an Fc-region they were purified by applying standard Protein A affinity chromatography. The antibodies were purified from cell culture supernatants by affinity chromatography using MabSelectSure-Sepharose™ (GE Healthcare, Sweden) and Superdex 200 size exclusion (GE Healthcare, Sweden) chromatography.

Briefly, sterile filtered cell culture supernatants were captured on a MabSelectSuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM sodium citrate at pH 3.0. The eluted antibody fractions were pooled and neutralized with 2 M Tris, pH 9.0. The antibody pools were further purified by size exclusion chromatography using a Superdex 200 26/60 GL (GE Healthcare, Sweden) column equilibrated with 20 mM histidine, 140 mM NaCl, pH 6.0. The 2/3-IgG containing fractions were pooled, concentrated to the required concentration using Vivaspin ultrafiltration devices (Sartorius Stedim Biotech S. A., France) and stored at −80° C.

Product purity was >98% for both, the first and the second fusion polypeptide targeting HER2. Samples were kept at 4° C. for >14 days to monitor stability and potential aggregate formation and found to be stable.

Stability of non-specific precursor molecules with covalently attached exchange units after >14 days storage at 4° C. was determined by analytical size exclusion chromatography.

### Example 3: T Cell Activation Mediated by Recombinant Binding Proteins of the Invention, Wherein the First and the Second Fusion Polypeptide Bind to the Same Epitope on a Target Cell

Pairs of recombinant binding proteins according to the invention with a domain arrangement as illustrated in FIG. 1 were expressed as described in Example 2. Both fusion polypeptides comprised in the pair of recombinant proteins comprised the same antigen binding domain.

A first pair of recombinant binding proteins was provided, wherein the first fusion polypeptide and the second fusion polypeptide comprise antigen binding domains specifically binding to Her2 and the effector domain was an anti-CD3 antigen binding site (Table 1).

TABLE 1

| | light chain | heavy chain | heavy chain-scFab fusion |
|---|---|---|---|
| <Her2>pro<CD3>VH | SEQ ID NO: 01 | SEQ ID NO: 02 | SEQ ID NO: 03 |
| <Her2>pro<CD3>VL | SEQ ID NO: 01 | SEQ ID NO: 02 | SEQ ID NO: 04 |

Also, a second pair of recombinant binding proteins was provided, wherein the first fusion polypeptide and the second fusion polypeptide comprise antigen binding domains specifically binding to EGFR and the effector domain was an anti-CD3 antigen binding site (Table 2).

TABLE 2

| | light chain | heavy chain | heavy chain-scFab fusion |
|---|---|---|---|
| <EGFR>pro<CD3>VH | SEQ ID NO: 05 | SEQ ID NO: 06 | SEQ ID NO: 07 |
| <EGFR>pro<CD3>VL | SEQ ID NO: 05 | SEQ ID NO: 06 | SEQ ID NO: 04 |

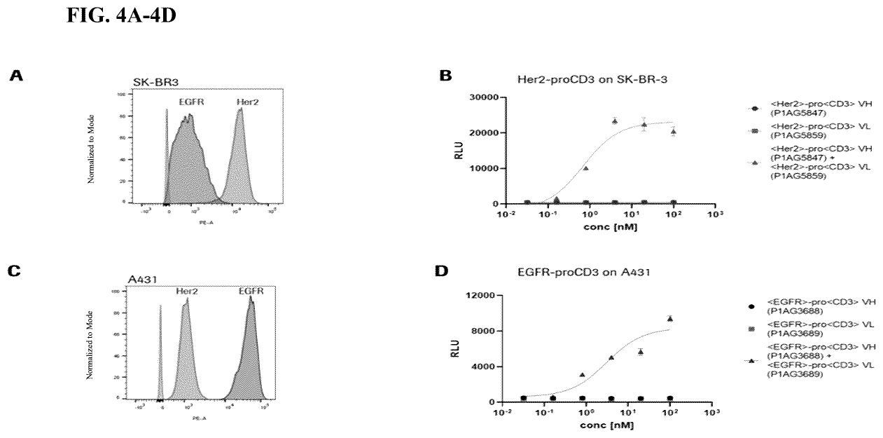

FIG. 4A shows the expression profile of the tumor associated antigens EGFR and HER2 on SK-BR3 and FIG. 4C shows the expression profile of the tumor associated antigens EGFR and HER2 on A431 cells.

In order to measure whether a functional effector domain specifically binding to CD3 is formed upon target cell binding, HER2-positive SK-BR3 cells were co-incubated with Jurkat reporter cells (E:T=1:3) according to the manufacturer's recommendations (Promega, #J1601) and respective antibodies targeting HER2. In short, 30.000 target cells were seeded out on day 1 into 96-well plates (flat white, clear bottom, Corning #3610). On day 2, medium was removed and 1×10\hat{ }5 Jurkat cells were added together with media and antibodies to reach a final volume of 75 μl. The HER2-targeted antibodies were included either alone (to measure the inactivation of the CD3-binder into a prodrug) or in combination (to measure the reconstitution of the CD3 binder) at equimolar concentrations. 16 hours post treatment, the recommended detection reagent was added and luminescence was measured using a TECAN microplate reader device.

FIG. 4B indicates activation of the effector domain in presence of the target cells and both, the first and the second fusion polypeptide, while no activity was observed when only one of the fusion polypeptides was present. Triplicate values were fitted with a 3-parameter non-linear regression and plotted using GraphPad Prism software.

In order to measure whether whether a functional effector domain specifically binding to CD3 is formed upon target cell binding by the second pair of recombinant binding proteins, EGFR-positive A431 cells were co-incubated with Jurkat reporter cells (E:T=1:3) according to the manufacturer's recommendations (Promega, #J1601) and respective antibodies targeting A431. FIG. 4D indicates activation of the effector domain in presence of the target cells and both, the first and the second fusion polypeptide, while no activity was observed when only one of the fusion polypeptides was present. Triplicate values were fitted with a 3-parameter non-linear regression and plotted using GraphPad Prism software. Triplicate values were fitted with a 3-parameter non-linear regression and plotted using GraphPad Prism software (D).

### Example 4: T Cell Activation Mediated by Recombinant Binding Proteins of the Invention, Wherein the First and the Second Fusion Polypeptide Bind to Different Epitopes on a Target Cell

Pairs of recombinant binding proteins according to the invention with a domain arrangement as illustrated in FIG. 1 were expressed as described in Example 2. Both fusion polypeptides comprised in the pair of recombinant proteins comprised the same antigen binding domain.

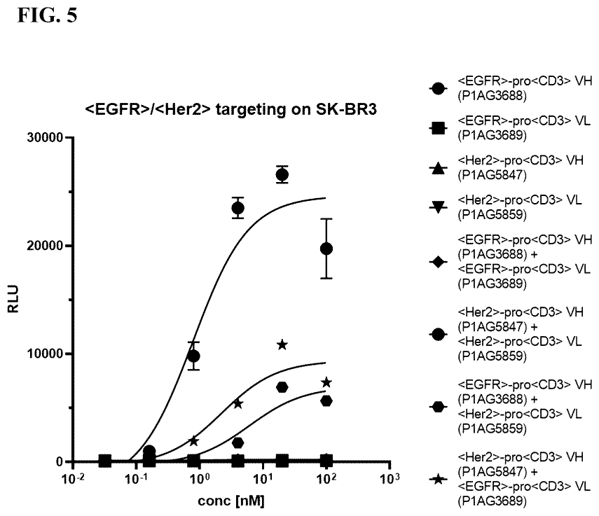

T cell activation mediated by different combinations of the recombinant binding proteins as used in example 3 was assessed using a SK-BR-3 cell line, which expresses both Her2 and EGFR. Briefly, HER2-, and EGFR-positive SK-BR3 cells were co-incubated with Jurkat reporter cells (E:T=1:3) according to the manufacturer's recommendations (Promega, #J1601) and respective fusion polypeptides targeting HER2 or EGFR. The fusion polypeptides were included either alone (control) or in combination (to assess the formation of the functional effector domain) at equimolar concentrations. 16 hours post treatment, the recommended detection reagent was added and luminescence was measured using a TECAN microplate reader device. Results are shown in FIG. 5, which indicates activation of the effector domain in presence of the target cells and both, the first and the second fusion polypeptide, while no activity was observed when only one of the fusion polypeptides was present. Triplicate values were fitted with a 3-parameter non-linear regression and plotted using GraphPad Prism software.

### Example 5 (Control): T Cell Activation Mediated by Recombinant Binding Proteins of the Invention, Wherein Only One of the Antigen Binding Domains of the First and Second Fusion Polypeptide Binds to an Epitope on a Target Cell

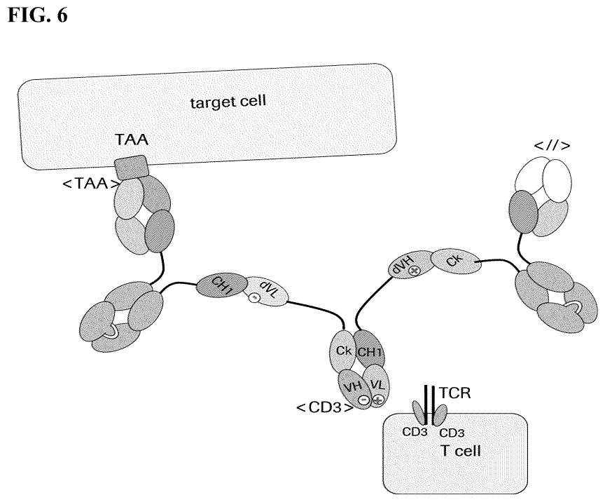

Pairs of recombinant binding proteins according to the invention with a domain arrangement as illustrated in FIG. 6 were expressed as described in Example 2 as a control.

FIG. 6 shows the principle of activating the effector domain upon binding of the first and the second fusion polypeptide on target cells via their first and second antigen binding domains, wherein only one of them binds to an epitope on a target cell, while the other antigen binding domain does not bind to a target on a surface cell describes one form of the undesired in-solution activation that needs to be minimized. The partially destabilized interfaces in the fusion polypeptides triggers the dissociation of the <CD3>-derived VH/VL chains. Driven by the attracting charges (VH,Q39E (Precursor A); VL, Q38K (Precursor B)) an active CD3-binding Fab is generated from precursors in close proximity and mediates the engagement and activation of T cells.

Pairs of recombinant binding proteins were provided, using first and second fusion polypeptides comprising an antigen binding domain specifically binding to Her2 and the effector domain was an anti-CD3 antigen binding site, as used in Example 3. Another set of fusion polypeptides was provided comprising an antigen binding domain that does not specifically bind to an epitope on a target cell (Table 3). As a control recombinant binding proteins of a regular IgG shape, i.e. with a regular Fab fragment instead of a single chain Fab fragment, were provided (Table 4).

TABLE 3

| | light chain | heavy chain | heavy chain-scFab fusion |
|---|---|---|---|
| <Nada>pro<CD3> VL (6G4S | SEQ ID NO: 07 | SEQ ID NO: 08 | SEQ ID NO: 04 |
| linker) | | | |

TABLE 4

| | light chain | heavy chain | heavy chain | light chain |
|---|---|---|---|---|
| <Her2>pro<CD3> VH (no linker) | SEQ ID NO: 01 | SEQ ID NO: 02 | SEQ ID NO: 09 | SEQ ID NO: 10 |
| <Her2>pro<CD3> VL (no linker) | SEQ ID NO: 01 | SEQ ID NO: 02 | SEQ ID NO: 11 | SEQ ID NO: 12 |
| <Nada>pro<CD3> VL (no linker) | SEQ ID NO: 07 | SEQ ID NO: 08 | SEQ ID NO: 11 | SEQ ID NO: 12 |

T cell activation mediated by different combinations of these recombinant binding proteins was assessed using a SK-BR-3 cell line, which expresses both Her2 and EGFR. Briefly, HER2-, and EGFR-positive SK-BR3 cells were co-incubated with Jurkat reporter cells (E:T=1:3) according to the manufacturer's recommendations (Promega, #J1601) and respective fusion polypeptides targeting HER2. The fusion polypeptides were included either alone (control) or in combination (to assess the formation of the functional effector domain) at equimolar concentrations. 16 hours post treatment, the recommended detection reagent was added and luminescence was measured using a TECAN microplate reader device.

Results using linker-free control recombinant binding proteins are shown in FIG. 7A, indicating an in-solution activation of the effector domain.

Results using recombinant binding proteins of the invention are shown in FIG. 7B, which indicates activation of the effector domain in presence of the target cells and both, the first and the second fusion polypeptide binding to the target cell, while almost no activity was observed when only one of the fusion polypeptides was present. Triplicate values were fitted with a 3-parameter non-linear regression and plotted using GraphPad Prism software.

### Example 6: Activation of a First CD3 Fab and a Second DIG Fab Mediated by Recombinant Binding Proteins of the Invention, Wherein the First and the Second Fusion Polypeptide Bind to the Same Epitope on a Target Cell

Pairs of recombinant binding proteins according to the invention with a domain arrangement as illustrated in FIG. 1 were expressed as described in Example 2.

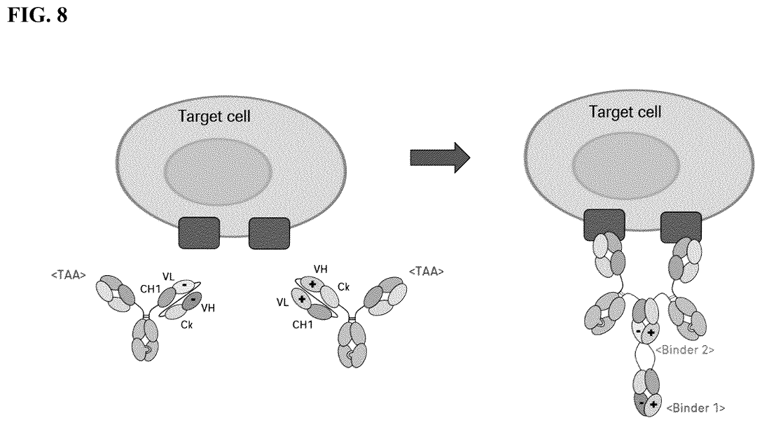

FIG. 8 illustrates the principle of activating two effector domains upon binding of the first and the second fusion polypeptide on target cells via their first and second antigen binding domains that bind to the same epitope on the surface of the target cell. The partially destabilized interfaces in the first and second fusion polypeptide triggers the dissociation of the VH/VL chains of the scFab fragments of the precursor molecules. Driven by the attracting charges (Binder 1: VH,Q39E (Precursor A); VL, Q38K (Precursor B)) or (Binder 2: VL,Q38E (Precursor A); VH,Q39K (Precursor B)), two active antigen-binding Fab fragments are generated from precursors in close proximity.

TABLE 5

| | light chain | heavy chain | heavy chain-scFab fusion |
|---|---|---|---|
| <Her2>(Dig)(CD3) [A] | SEQ ID NO: 01 | SEQ ID NO: 02 | SEQ ID NO: 13 |
| <Her2>(Dig)(CD3) [B] | SEQ ID NO: 01 | SEQ ID NO: 02 | SEQ ID NO: 14 |
| <Her2>(CD3)(Dig) [A] | SEQ ID NO: 01 | SEQ ID NO: 02 | SEQ ID NO: 15 |
| <Her2>(CD3)(Dig) [B] | SEQ ID NO: 01 | SEQ ID NO: 02 | SEQ ID NO: 16 |
| <Her2>(Nada)(CD3) [A] | SEQ ID NO: 01 | SEQ ID NO: 02 | SEQ ID NO: 03 |
| <Her2>(Nada)(CD3) [B] | SEQ ID NO: 01 | SEQ ID NO: 02 | SEQ ID NO: 04 |
| <Her2>(CD3)(Nada) [A] | SEQ ID NO: 01 | SEQ ID NO: 02 | SEQ ID NO: 17 |
| <Her2>(CD3)(Nada) [B] | SEQ ID NO: 01 | SEQ ID NO: 02 | SEQ ID NO: 18 |

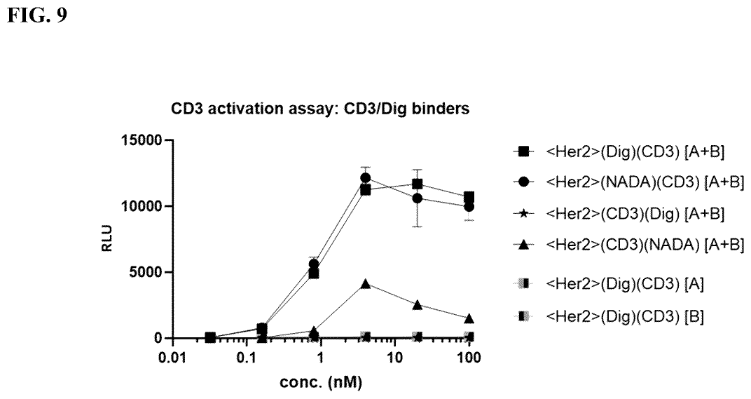

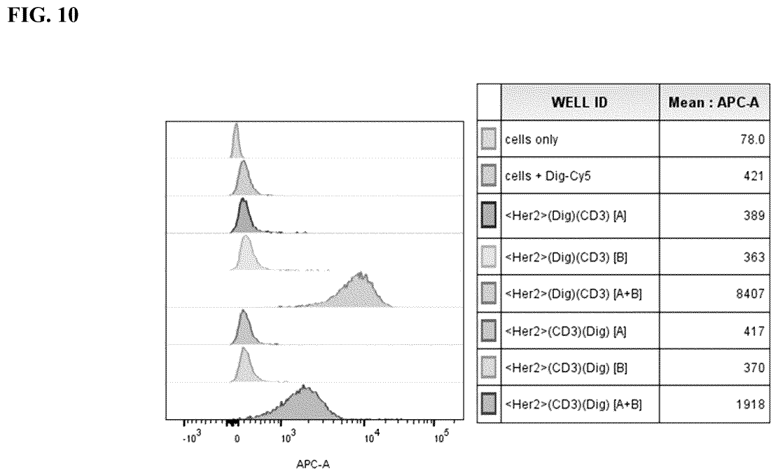

In order to demonstrate, that two functional effector domains may be formed using the concept of the invention, pairs of recombinant binding proteins were provided (Table 5), using first and second fusion polypeptides comprising an antigen binding domain specifically binding to Her2, and a first effector domain was an anti-CD3 antigen binding moiety (see FIG. 8 “Binder 1”) while a second effector domain was a a Digoxigenin (Dig)-binding entity (see FIG. 8 “Binder 2”). Combining VH or VL of the CD3 binder with VL or VH of the Dig-binder in the first and second fusion polypeptide rendered both binding entities nonfunctional. Targeted chain exchange on Her2-expressing SK-BR-3 cells activated both binding entities, generating a CD3-binding as well as a Dig-binding entity on the surface of target cells. Dependent on the position of the CD3- and Dig-variable-regions, CD3 and Dig-binding functionalities of the cell-bound prodrugs are positioned either proximal to the IgG-hinge-region (herein defined as ‘inner binder’), or distal, i.e. at the N-termini of the products (herein defined as ‘outer binder’). The nomenclature of the molecules in FIG. 9 and FIG. 10 is as follows: <Target> (Inner binder) (Outer binder) [Prodrug A/B]. Functionality of the CD3 binder was demonstrated in T cell activation assays of the different combinations of these recombinant binding proteins assessed using a SK-BR-3 cell line, which expresses Her2 as described in the examples above. Formation of functional Dig-binders was assessed by detecting Dig-Binding functionality on cells with fluorescence-labeled Digoxigenin. The results of these experiments confirm that the prodrugs assemble on target cells into products that bind CD3 as well as Dig (FIG. 9 and FIG. 10). The results also show that it is possible to generate active CD3 binders and active Dig binders in the inner and also in the outer position.

### Example 7: Activation of a First CD3 Fab and a Second CD28 Fab Mediated by Recombinant Binding Proteins of the Invention, Wherein the First and the Second Fusion Polypeptide Bind to the Same Epitope on a Target Cell

In order to assess CD28-mediated co-stimulation of T cells, four sets of HER2-targeted pairs of recombinant binding proteins according to the invention with a domain arrangement as illustrated in FIG. 8 were expressed as described in Example 2. Co-stimulation of T cells through CD28 signaling, also known as signal 2 in the T cell activation pathway, is dependent on initial T cell receptor activation, such as through CD3 crosslinking (PMID 23470321).

In the first two pairs of recombinant binding proteins, which were prepared as controls, CD3 VH/VL domains were paired with non-binding VH/VL “nada” dummy domains, such that chain-exchange results in a productive CD3 binder in either the inner or the outer positions.

In the other two pairs of recombinant binding proteins, CD3 VH/VL domains were paired with CD28 VH/VL domains, such that chain-exchange results in productive CD3 and CD28 binders in either the inner or the outer position. The CD28 binder used in this example is derived from TGN1412, clone 5.11A (PMID 12707299, US20040092718A1). The CD3 binder used in this example was 40G5c (U.S. Pat. No. 10,174,124B2).

The following molecules were used (Table 6):

In one set of recombinant binding proteins, the first fusion polypeptide comprises antigen-binding domains specifically binding to HER2, and the second fusion polypeptide comprises the effector domain, consisting of half of an anti-CD3 antigen-binding site in the “outer position” paired with either half of a Nada or CD28 binding site in the “inner position.”

TABLE 6

| | light chain | heavy chain | heavy chain-scFab fusion |
|---|---|---|---|
| <HER2>(NADA)(CD3) [A] | SEQ ID NO: 01 | SEQ ID NO: 02 | SEQ ID NO: 03 |
| <HER2>(NADA)(CD3) [B] | SEQ ID NO: 01 | SEQ ID NO: 02 | SEQ ID NO: 04 |
| <HER2>(CD28)(CD3) [A] | SEQ ID NO: 01 | SEQ ID NO: 02 | SEQ ID NO: 19 |
| <HER2>(CD28)(CD3) [B] | SEQ ID NO: 01 | SEQ ID NO: 02 | SEQ ID NO: 20 |

In another set of recombinant binding proteins (Table 7), the first fusion polypeptide comprises antigen-binding domains specifically binding to HER2, and the second fusion polypeptide comprises the effector domain, consisting of half of an anti-CD3 antigen-binding site in the “inner position” paired with either half of a NADA or CD28 binding site in the “outer position”.

TABLE 7

| | light chain | heavy chain | heavy chain-scFab fusion |
|---|---|---|---|
| <HER2>(CD3)(NADA) [A] | SEQ ID NO: 01 | SEQ ID NO: 02 | SEQ ID NO: 21 |
| <HER2>(CD3)(NADA) [B] | SEQ ID NO: 01 | SEQ ID NO: 02 | SEQ ID NO: 22 |
| <HER2>(CD3)(CD28) [A] | SEQ ID NO: 01 | SEQ ID NO: 02 | SEQ ID NO: 23 |
| <HER2>(CD3)(CD28) [B] | SEQ ID NO: 01 | SEQ ID NO: 02 | SEQ ID NO: 24 |

In order to assess CD28 signaling, HER2-positive SK-BR-3 cells were co-cultured together with Jurkat IL-2 promoter cells (Promega, catalog no. J1631). These engineered Jurkat T cells express low levels of luciferase in response to T cell activation through CD3-mediated TCR clustering. Upon CD28 co-stimulation, they express a greater level of luciferase. Thus, these reporter cells integrate both CD3 and CD28 signals into one overall luminescence readout. By comparing the signal obtained in the control CD3/nada constructs to that of the dual activated CD3/CD28 constructs, CD28 signaling could be assessed.

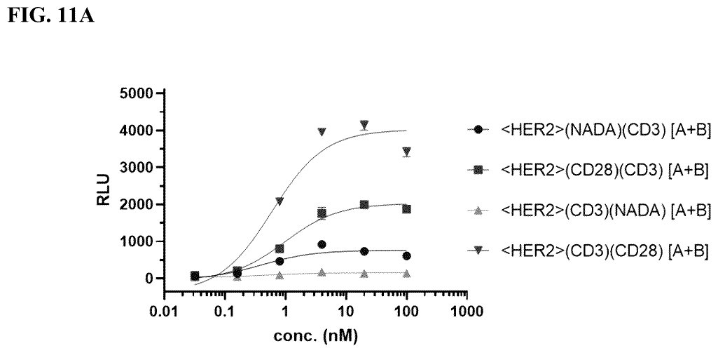

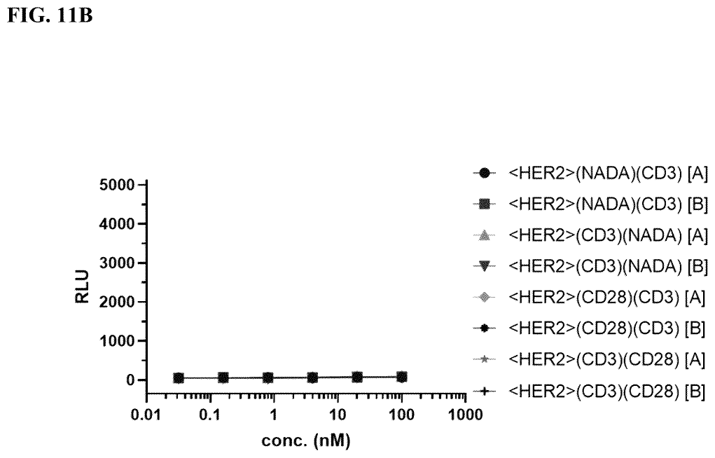

An example of T cell co-stimulation through simultaneous CD28 receptor activation and CD3 crosslinking is shown in FIG. 11A. CD28-mediated co-stimulation of T cells was observed with both recombinant binding pairs according to the invention, both with CD28 in the inner and in the outer position. The co-stimulation was dependent on the presence of both fusion polypeptides, and therefore on chain-exchange, as the individual fusion proteins alone without the presence of their respective counterpart did not lead to any T-cell activation (FIG. 11B).

These results demonstrate that the recombinant binding proteins according to the invention can be utilized to activate two antigen binding functionalities on a target cell.

### Example 8: Activation of a First IL2-Ry Fab and a Second IL-2RB Fab Mediated by Recombinant Binding Proteins of the Invention, Wherein the First and the Second Fusion Polypeptide Bind to the Same Antigen on a Target Cell

Further precursor molecules were prepared in order to demonstrate that the domain arrangement applied in the recombinant binding proteins of the invention is universally applicable independent of the antigen binding specificities.

Another application of targeted prodrug approaches that activate two functionalities is antibody-mediated activation of cell surface receptors. For certain receptors, ligand-induced activation of intracellular pathways is conferred by structural changes and/or hetero-dimerization of receptor subunits on cell surfaces. Such events that are naturally triggered by the respective receptor ligands can in some instances also be achieved by antibodies that connect and/or modulate receptor subunits. To mimic ligand functionality, receptor-binding antibodies or bispecifics must bind suitable positions in suitable receptor subunits in suitable formats. Because of this complexity, identifying molecules that fulfill all these parameters and thereby trigger receptor signaling is very challenging

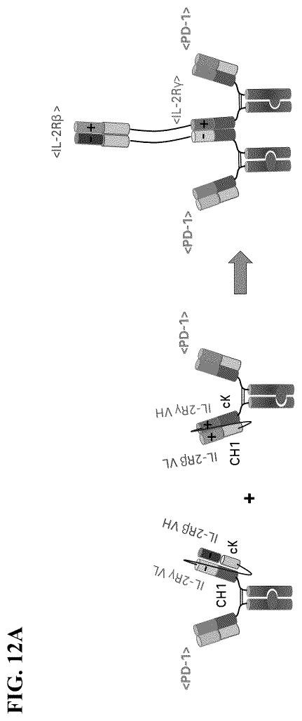

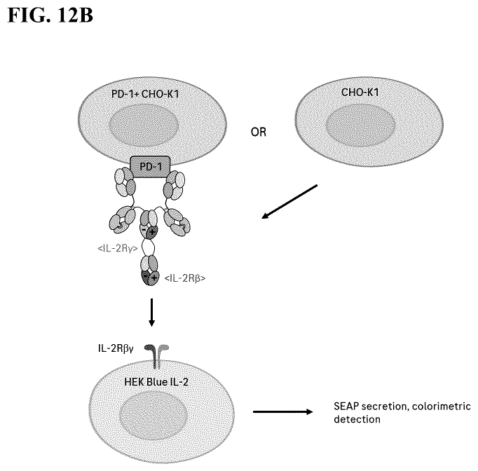

Thus, in another example, recombinant binding proteins capable of forming two effector domains, directed to IL2-Rγ and IL-2Rβ respectively, were generated. The overall arrangement of the recombinant binding proteins was as illustrated in FIG. 8 and FIG. 12A.

An illustration of the recombinant binding proteins that undergo chain exchange to activate the beta and gamma subunits of the IL-2R used in this example is shown in FIG. 12A. PD1-binders were used as antigen binding domains to enable targeted cell-surface accumulation of the precursor molecules.

For these molecules, the IL-2Rβ binders applied were Mik-β1 (J Immunol. 1993 Jul. 15; 151(2): 1075-85) and Cl.4 (in house), and the IL-2Rγ binders were Cl.3 and Cl.1 (both in house). VH-VL prodrug combinations derived from these binders encompassed Mik-β1(VH)-Cl.3(VL), Cl.4(VH)-Cl.1(VL), Cl.4(VH)-Cl.3(VL), and as complementary counterparts Mik-β1(VL)-Cl.3(VH), Cl.4(VL)-Cl.1(VH), Cl.4(VL)-Cl.3(VH). The targeting arm of each complementary set of prodrugs contained either the PD-1 binder 0376 (heavy chain of SEQ ID NO:25, light chain of SEQ ID NO:26) or the PD-1 binder 1040 (heavy chain of SEQ ID NO:30, light chain of SEQ ID NO:31), which bind to two different epitopes on PD-1.

Pairs of recombinant binding proteins according to the invention with a domain arrangement as illustrated in FIG. 12A were expressed as described in Example 2. Each knob-containing heavy chain comprised in the pair of recombinant proteins comprised a different PD-1 antigen binding domain.

A first set of recombinant binding proteins was provided (prodrug A), wherein the first fusion polypeptide and the second fusion polypeptide comprise antigen binding domains specifically binding to a distinct epitope on human PD-1, and the effector domain was a split anti-IL-2Rβ binder in the outer position and a split anti-IL-2Rγ binder in the inner position.

The following molecules were used (Table 8):

TABLE 8

| | | | heavy chain- |
|---|---|---|---|
| Name | light chain | heavy chain | scFab fusion |
| XVV142 | SEQ ID NO: 25 | SEQ ID NO: 26 | SEQ ID NO: 27 |
| XVV143 | SEQ ID NO: 25 | SEQ ID NO: 26 | SEQ ID NO: 28 |
| XVV144 | SEQ ID NO: 25 | SEQ ID NO: 26 | SEQ ID NO: 29 |

Also, a second set of recombinant binding proteins was provided (prodrug B), wherein the first fusion polypeptide and the second fusion polypeptide comprise antigen binding domains specifically binding to a second distinct epitope on human PD-1, and the effector domain was a complementary split anti-IL-2Rβ binder in the outer position and a complementary split anti-IL-2Rγ binder in the inner position.

The following molecules were used (Table 9):

TABLE 9

| | | | heavy chain- |
|---|---|---|---|
| Name | light chain | heavy chain | scFab fusion |
| XVV146 | SEQ ID NO: 30 | SEQ ID NO: 31 | SEQ ID NO: 32 |
| XVV147 | SEQ ID NO: 30 | SEQ ID NO: 31 | SEQ ID NO: 33 |
| XVV148 | SEQ ID NO: 30 | SEQ ID NO: 31 | SEQ ID NO: 34 |

In order to assess formation of functional IL-2R agonists upon accumulation on PD-1-expressing cells, a HEK-Blue IL-2 transactivation assay was performed upon co-incubation of the precursor molecules with PD-1-expressing CHO-K1 cells or with PD-1-negative CHO-K1 control cells (for a schematic of the assay, see FIG. 12B).

Briefly, 2.5×10⁴ CHO-K1 cells overexpressing human PD-1 or 2.5×10⁴ parental CHO-K1 cells were seeded out in flat-bottom 96-well plates. The following day, 5×10⁴ HEK-Blue IL-2 reporter cells (InvivoGen) were added to the plates followed by serial dilutions of the respective precursor molecules. The plates were incubated for 22 h at 37° C., 5% CO₂, and 80% relative humidity. Following incubation, 20 μl of supernatant was added to 180 μl of QuantiBlue solution (InvivoGen, rep-qbs2), and the reaction was incubated for 45 min at 37° C. IL-2R activation was quantified by measuring the absorbance at 640 nm using a Tecan Infinite F200 Pro plate reader. The data were analyzed and fit with a three-parameter, non-linear regression using GraphPad Prism software.

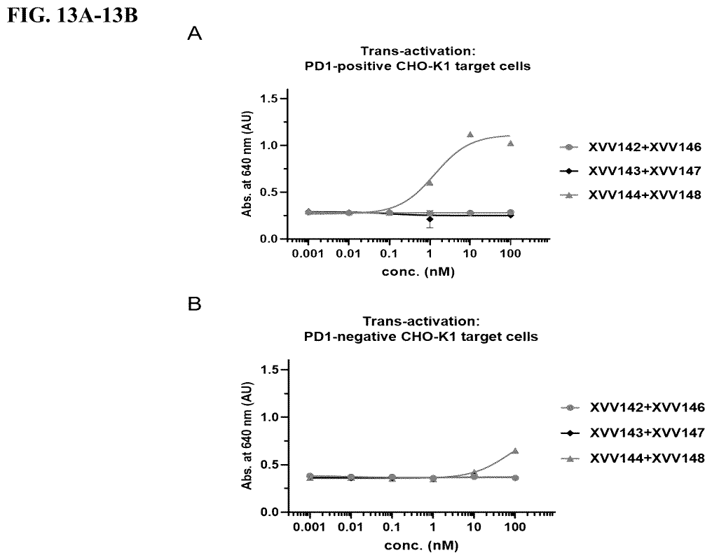

The results of these analyses are shown in 13. Inactive PD-1-targeted precursor molecules (regarding IL-2R activation) can be converted into active IL-2 receptor agonists upon accumulation on PD-1-expressing cells (FIG. 13A). FIG. 13B shows that prodrug activation is target-specific because co-incubation with parental CHO-K1 cells that do not express PD-1 generated almost no IL-2R-dependent signals under otherwise identical conditions.

1. A pair of recombinant binding proteins, comprising
(a) a first fusion polypeptide comprising (i) a first antigen binding domain capable of binding to a target antigen, (ii) a first part of an effector domain, and (iii) a first complementing domain capable of association with the first part of the effector domain, wherein the first part of the effector domain and the first complementing domain are connected via a peptide linker; and
(b) a second fusion polypeptide comprising (i) a second antigen binding domain capable of binding to a target antigen, (ii) a second part of an effector domain, and (iii) a second complementing domain capable of association with the second part of the effector domain, wherein the first part of the effector domain and the first complementing domain are connected via a peptide linker;
wherein the first and the second part of the effector domain are capable of associating with each other to form a functional effector domain, characterized in that the effector domain is an antibody Fab fragment.

2. The pair of recombinant binding proteins according to claim 1,
wherein the first part of the effector domain is an antibody heavy chain polypeptide and the first complementing domain is an antibody light chain polypeptide, and
wherein the second part of the effector domain is an antibody light chain polypeptide and the second complementing domain is an antibody heavy chain polypeptide.

3. The pair of recombinant binding proteins according to one of the preceding claims, wherein the first part of the effector domain comprises a variable heavy chain domain having a Q39E mutation, and wherein the first complementing domain comprises a variable light chain domain having a Q38E mutation,
wherein the second part of the effector domain comprises a variable light chain domain having a Q38K mutation, and wherein the second complementing domain comprises a variable heavy chain domain having a Q39K mutation.

4. The pair of recombinant binding proteins according to one of the preceding claims, wherein the first part of the effector domain and the first complementing domain are comprised in a single chain Fab fragment, and wherein the second part of the effector domain and the second complementing domain are comprised in a single chain Fab fragment.

5. The pair of recombinant binding proteins according to one of claim 5 or 6, wherein in the single chain Fab fragment of the first fusion protein the VH and VL domain are exchanged with each other and wherein in the single chain Fab fragment of the second fusion protein the CH1 and CL domains are exchanged with each other.

6. The pair of recombinant binding proteins according to one of claim 5 or 6, wherein the single chain Fab fragment of the first fusion protein comprises from N- to C-terminal direction a VH domain, a CL domain, a peptide linker, a VL domain and a CH1 domain, and wherein the single chain Fab fragment of the second fusion protein comprises from N- to C-terminal direction a VL domain, a CH1 domain, a peptide linker, a VH domain and a CL domain.

7. The pair of recombinant binding proteins according to one of the preceding claims, wherein the first antigen binding domain is an antibody Fab fragment.

8. The pair of recombinant binding proteins according to one of the preceding claims, wherein the second antigen binding domain is an antibody Fab fragment.

9. The pair of recombinant binding proteins according to one of the preceding claims, wherein the first fusion polypeptide and the second fusion polypeptide comprise a (heterodimeric) Fc domain.

10. The pair of recombinant binding proteins according to one of the preceding claims, wherein the first fusion polypeptide as well as the second fusion polypeptide comprise a heterodimeric Fc domain comprising two CH3 domains, wherein one of the CH3 domains comprises the mutations S354C and T366W, and the other CH3 domain comprises the mutations Y349C, L368A and Y407V.

11. The pair of recombinant binding proteins according to one of the preceding claims, wherein the first fusion polypeptide is a recombinant antibody consisting of three polypeptides: (i) an antibody light chain, and (ii) an antibody heavy chain, wherein the variable domains of said antibody light chain and said antibody heavy chain form the first antigen binding domain, and (iii) an antibody heavy chain/light chain fusion polypeptide, comprising an antibody light chain comprising the first part of the effector domain fused via a peptide linker to an antibody heavy chain comprising the first complementing domain, wherein the C-terminus of the antibody light chain is fused to the N-terminus of the antibody heavy chain; and
wherein the second fusion polypeptide is a recombinant antibody consisting of three polypeptides: (i) an antibody light chain, and (ii) an antibody heavy chain, wherein the variable domains of said antibody light chain and said antibody heavy chain form the second antigen binding domain, and (iii) an antibody heavy chain/light chain fusion polypeptide, comprising an antibody light chain comprising the second part of the effector domain fused via a peptide linker to an antibody heavy chain comprising the second complementing domain, wherein the C-terminus of the antibody light chain is fused to the N-terminus of the antibody heavy chain.

12. The pair of recombinant binding proteins according to one of the preceding claims, wherein the effector domain is antibody binding domain.

13. The pair of recombinant binding proteins according to one of the preceding claims comprising two effector domains.

14. The pair of recombinant binding proteins according to one of the preceding claims, wherein the effector domain specifically binds to an activating T cell antigen.

15. The pair of recombinant binding proteins according to claim 13 or 14, wherein the the recombinant binding protein comprises two effector domains, wherein the two effector domains specifically binds to two different T cell antigens.

16. A method for forming a functional effector domain from a pair of recombinant binding proteins according to one of the preceding claims, wherein the first antigen binding domain and the second antigen binding domain of the recombinant binding proteins specifically bind to an epitope on a surface of a target cell, the method comprising contacting the pair of recombinant binding proteins with a target cell under conditions allowing the binding of the first fusion polypeptide and the second fusion polypeptide to the target cell.

17. A pharmaceutical formulation comprising the pair of recombinant binding proteins according to any one of the preceeding claims and a pharmaceutically acceptable carrier.
